(19) United States
(12) Patent Application Publication
(10) Pub. No.: US 2010/0178260 A1
(43) Pub. Date: Jul. 15, 2010

(54) COMPOSITION COMPRISING A RETINOID, AN ADENOSINE-BASED NONPHOSPHATED COMPOUND, AND A SEMICRYSTALLINE POLYMER

(75) Inventors: Danielle RAVAUX, Saint-Arnoult En Yvelines (FR); Angeles Fonolla MORENO, Paris (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: L'OREAL, Paris (FR)

(21) Appl. No.: 12/687,225

(22) Filed: Jan. 14, 2010

Related U.S. Application Data
(60) Provisional application No. 61/149,346, filed on Feb. 3, 2009.

(30) Foreign Application Priority Data
Jan. 15, 2009 (FR) 09 50238

(51) Int. Cl. A61K 8/67; A61Q 17/04; A61K 8/72

(52) U.S. Cl. 424/59; 424/78.03

(57) ABSTRACT

Composition containing, in a physiologically acceptable medium, at least one retinoid or a derivative thereof, one adenosine-based nonphosphated compound, and one semicrystalline polymer with one or more crystallizable side chain(s). Use for preventing and/or treating the cutaneous signs of ageing.

### REFERENCE TO PRIOR APPLICATIONS

This application claims priority to U.S. provisional application Ser. No. 61/149,346, filed Feb. 3, 2009; and to French patent application 09 50238, filed Jan. 15, 2009, both incorporated herein by reference.

### FIELD OF THE INVENTION

The present invention relates to compositions, preferably cosmetic or dermatological compositions, comprising a combination of active agents for combating the cutaneous signs of ageing, and in particular combating wrinkled skin and/or sagging skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

The concern to preserve for as long as possible a young-looking skin is a preoccupation of the majority of women and is also increasingly affecting the men. In order to meet this expectation, cosmetic compositions aimed at preventing and/or treating the signs of skin ageing have therefore been developed.

Skin ageing is defined by all the alterations of the cutaneous covering resulting from the accumulation over the years of the gradual modifications of its various constituents.

It results, inter alia, in a flattening of the epidermis and of the dermo-epidermal junction and in the dermis, through, on the one hand, an overall reduction in the extracellular matrix (ECM) associated with a gradual decrease in collagen and elastin fibre production by fibroblasts and, on the other hand, by an increase in the destruction of these macromolecules by specific enzymes.

These biological phenomena therefore result in the induction, in the skin, of considerable physical modifications: loss of firmness, sagging. The skin loses its elasticity and the features slacken. The slackening of the subcutaneous tissues (fats and muscles) leads to an excess of skin and ptosis. This slackening is characterized by drooping of the cheekbones and of the cheeks, entraining the lower eyelid. Associated with surface mechanical stress, wrinkles form then accentuate and become deeper. In addition, during the day, depending on water movement due to gravity, some wrinkles increase and become more marked.

Among the wrinkles that coexist on the face, it is thus possible to make a distinction between embryonic wrinkles which are derived from small starting points invisible to the naked eye, which join up together over time to form a wrinkle; deeper marked wrinkles, resulting from the hollowing of certain furrows over time; and reversible wrinkles, which originate from the decrease over the day in the thickness of the skin and in the increase in its elasticity.

A certain number of compounds have already been identified as antiwrinkle active agents and used in cosmetic compositions for the purposes of combating the cutaneous signs of ageing, and in particular decreasing and/or smoothing out skin wrinkles.

For example, retinol has a certain efficacy as an antiwrinkle active agent, in particular by virtue of its antidifferentiating properties. However, retinol is an active agent which only acts longterm. Moreover, in order to ensure tolerance in cosmetics, the amount of retinol introduced into cosmetic compositions is restricted, thereby limiting its effectiveness.

In order to partly make up fir these insufficiencies, combinations of retinol with other components have already been proposed, and in particular with other active agents recognized for their antiwrinkle activity, such as adenosine, known for its “retinol-like” activity with respect to the TGK enzyme. Thus, patent application JP 10007541 has proposed a lotion for combating the effects on the skin associated with oxidative stress, and in particular wrinkles, comprising in particular vitamin A (retinol) and adenosine.

The use of adenosine derivatives in cosmetic compositions for the purposes of combating wrinkles, in particular expression wrinkles, and/or decontracting the skin and/or relaxing the features of the skin is also known from application EP 1 847 547. The addition of various additional compounds such as retinol and derivatives thereof is envisaged.

However, the effectiveness of combinations of this type is not always immediate. In other words, it will generally correspond to only medium-term or long-term treatment of wrinkles. The visible effect of these compositions thus occurs only after a certain application time, which may range from several days to several weeks.

The invention aims precisely to compensate for this insufficiency, and makes it possible in particular to accelerate the manifestation of a visible effect on the reducing and/or smoothing out of skin wrinkles.

### BRIEF DESCRIPTION OF THE DRAWING

FIG. 1 shows the results of Realskin epidermises incubated in the presence or absence of active agents.

### DETAILED DESCRIPTION OF THE PREFERRED EMBODIMENTS

According to one of its first aspects, the invention relates to a composition, prefereably a cosmetic or dermatological composition, comprising, in a physiologically acceptable medium, at least one retinoid or a derivative thereof, one adenosine-based nonphosphated compound and one semicrystalline polymer with one or more crystallizable side chain(s). Another preferred aspect of the invention is at least one combination comprising at least one retinoid or a derivative thereof, one adenosine-based nonphosphated compound and one semicrystalline polymer with one or more crystallizable side chain(s). In the following description, where a composiotn is mentioned a combination is included, and vice versa.

WO 2007/141142 describes the use of a semicrystalline polymer, in particular a semicrystalline polymer with one or more crystallizable chain(s), in combination with a 2-acrylamido-2-methylpropanesulphonic acid polymer comprising at least one hydrophobic unit, in O/W emulsions. Such a combination allows the formulation of stable O/W emulsions which are advantageously free of surfactants, reconciling the advantages provided by the presence of fatty substances such as waxes, which are beneficial for nourishing the skin, with a pleasant texture, easy application and a feeling of freshness on application. The cosmetic compositions incorporating such a combination may, for example, be care products for treating wrinkles and the signs of skin ageing, moisturizing the skin or treating sensitive or dry skin. However, it is in no way envisaged in this document that a semicrystalline polymer with one or more crystallizable side chain(s) may be used in itself, in combination with at least one retinoid or a derivative thereof and at least one adenosine-based nonphosphated compound, for the purposes of reinforcing the antiwrinkle effectiveness of cosmetic or dermatological compositions.

The inventors have noted that a combination of active agents according to the invention makes it possible to effectively combat the signs of skin ageing associated with wrinkled or sagging skin, and in particular to effectively fade out wrinkles, with an immediate visible effect, and a reinforced effect in the short-, medium- and long-term.

Against all expectations, the inventors have thus noted that the presence of a semicrystalline polymer with one or more crystallizable side chain(s) makes it possible to obtain a structuring of the composition that is particularly advantageous with regard to the wrinkle-filling and -resurfacing effect, in particular desired according to the invention, and also intrinsic soft-focus effects (haziness, mattness, etc.) which, in parallel, make it possible to cloud the visual perception of the wrinkles. This results in a virtually immediate camouflage effect on the wrinkles which, by manifesting itself together with the antiwrinkle effect of the retinoid and adenosine combination, makes it possible to significantly reinforce the antiwrinkle effectiveness of the compositions according to the invention with regard to already existing compositions.

Such a combination of active agents makes it possible in particular to act effectively on the texture of the skin, and to provide in particular skin which is smoother, more homogeneous and firmer, which has more tonicity and which is more elastic.

Thus, according to another of its aspects, the present invention relates to the use, in particular cosmetic use, of a composition and/or combination as described above, for preventing and/or treating the signs of skin ageing.

The signs of skin ageing are in particular a loss of firmness, of density, of elasticity and of tonicity of the skin, thinning of the epidermis, sagging, slackening of cutaneous and/or subcutaneous tissues, of ptosis type for example, fine lines and wrinkles.

The compositions according to the invention are more particularly advantageous for preventing and/or treating the cutaneous signs of ageing, in particular as defined above, and more particularly chosen from wrinkled skin and/or sagging skin.

Thus, according to yet another of its aspects, the present invention relates to the use of a combination as described above, for preventing and/or treating wrinkled skin and/or sagging skin.

Such a combination is especially Particularly effective for decreasing and/or smoothing out embryonic and/or deep and/or reversible wrinkles, as defined above.

The wrinkles concerned may, for example, be those lying radially around the mouth and/or the eyes, in particular crowsfeet wrinkles, wrinkles under the eye and/or on the forehead, in particular the “lion” wrinkle, located on the glabella, in the space between the eyebrows, and/or lying horizontally on the forehead, wrinkles of the nasal groove, and ptosis of the lower part of the face.

According to another of its aspects, the invention is directed towards a nontherapeutic or cosmetic treatment method for the skin, in particular human skin, intended to prevent and/or treat the cutaneous signs of ageing, in particular wrinkles, comprising at least the application, to the skin, of a combination and/or of a composition as defined above.

Advantageously, the composition according to the invention is intended to be applied to the areas of the face and/or of the forehead which are marked by wrinkles.

The method of the invention is more particularly intended to be implemented for individuals exhibiting signs of skin ageing, and in particular individuals with wrinkled and/or sagging skin.

According to one embodiment of the invention, the combination under consideration according to the invention may be used with a secondary anti-ageing, and in particular antiwrinkle, active agent.

For the purpose of the present invention, the term “antiwrinkle active agent” is intended to mean a compound of natural or synthetic origin which produces a biological effect, such as an increase in the synthesis and/or the activity of certain enzymes, when it is brought into contact with an area of wrinkled skin, this effect having the result of reducing the appearance of wrinkles and/or fine lines.

The compositions that may be used in the context of the present invention may be cosmetic, pharmaceutical and/or dermatological compositions, and more particularly are cosmetic compositions.

For the purpose of the present invention, the term “a cosmetic composition” denotes a composition capable of producing an effect on the skin in terms of attractiveness or comfort, also for beauty purposes, for example with a view to protecting it, to keeping it in good condition, to modifying the appearance thereof and in particular to making it more attractive.

According to one variant of the invention, the composition is more particularly a composition for topical application.

### Retinoids

The retinoid and retinoid derivatives according to the invention include retinol (vitamin A), retinal (vitamin A aldehyde), retinoic acid (vitamin A acid), or an ester of retinol and of a C₂-C₂₀ acid, such as the propionate, the acetate, the linoleate or the palmitate of retinol (retinyl palmitate), among others.

Among the retinoids, mention may more particularly be made of retinol, retinal, retinoic acid, in particular all-trans retinoic acid and 13-cis retinoic acid, retinol derivatives, such as retinyl acetate, propionate or palmitate, and the retinoids described in the following patent applications: FR 2 370 377, EP 0 199 636. EP 0 325 540 and EP 0 402 072:

According to one preferred embodiment of the invention, the retinoid is retinol or a proretinol.

The term “retinol” is intended to mean all the isomers of retinol, i.e. all-trans retinol, 13-cis retinol, 11-cis retinol, 9-cis retinol and 3,4-didehydro retinol.

As a representative of proretinol compounds, mention may in particular be made of retinyl palmitate (the palmitate of retinol).

Most particularly suitable are those sold by the company BASF.

According to one embodiment, the retinoid may be present in a composition of the invention in a proportion of approximately 0.005% to 5% by weight, preferably of approximately 0.01% to 2% by weight. and in particular of approximately 0.05% to 0.5% by weight, relative to the total weight of the composition.

### Adenosine-Based Nonphosphated Compounds

A composition of the invention comprises at least one adenosine-based nonphosphated compound.

This expression is intended to mean both adenosine itself and its nonphosphated derivatives.

By way of examples of nonphosphated derivatives of adenosine, mention may be made of: adenosine; 2′-deoxyadenosine; 2′,3′-isopropylidene adenosine, toyocamycin; 1-methyladenosine, N-6-methyladenosine; adenosine N-oxide; 6-methylmercaptopurine riboside; and 6-chloropurine riboside.

Other adenosine derivatives comprise adenosine receptor agonists, including phenylisopropyladenosine (PIA), 1-methylisoguanosine, N⁶-cyclohexyladenosine (CHA), N⁶-cyclopentyladenosine (CPA), 2-chloro-N6-cyclopentyladenosine, 2-chloroadenosine, N⁶-phenyladenosine, 2-phenylaminoadenosine, MECA, N⁶-phenethyladenosine, 2-p-(2-carboxyethyl)phenethylamino-5′-N-ethylcarboxamidoadenosine (CGS-21680), N-ethyl-carboxamidoadenosine (NECA), 5′-(N-cyclopropyl)carboxamidoadenosine, DPMA (PD 129,944) and metrifudil.

Other adenosine derivatives comprise compounds which increase the intracellular adenosine concentration, such as erythro-9-(2-hydroxy-3-nonyl)adenine (EHNA) and iodotubercidin.

The adenosine derivatives described in application EP 1 847 547 are also incorporated by way of reference.

Yet other derivatives of adenosine comprise the salts and the alkyl esters thereof

Adenosine is preferred for use in the present invention. It is in particular commercially available in powder form from the company Pharma Waldhof.

In the compositions in accordance with the invention, the adenosine-based compound preferably represents from 0.0001% to 5% by weight, more preferably from 0.001% to 1% by weight, and better still from 0.01% to 0.5% by weight, relative to the total weight of the composition.

Semicrystalline Polymers with One or More Crystallizable Side Chain(S)

A composition according to the invention comprises at least one semicrystalline polymer with one or more crystallizable side chain(s), preferably chosen from C₁₀-C₃₀ alkyl (meth)acrylate homopolymers.

As specified above, the presence of such a polymer is in particular advantageous from the viewpoint of the texturing effect that it gives to the composition. The latter makes it possible in fact to provide an immediate smoothing effect on the skin to which it is applied.

This texturing is more particularly carried out at the level of the fatty phase of the composition, in which said polymer is formulated.

For the purpose of the invention, the term “polymers” is intended to mean compounds comprising at least two repeating units, preferably at least three repeating units, and more especially at least ten repeating units.

For the purpose of the invention, the term “semicrystalline polymer” is intended to mean polymers comprising at least one crystallizable pendent chain having a temperature for reversible phase change of the first order, in particular melting point (solid-liquid transition).

The term “crystallizable chain” is intended to mean a chain comprising at least 10 carbon atoms, and which, if it was alone, would change reversibly from the amorphous state to the crystalline state, depending on whether or not the temperature is above or below the melting point. For the purpose of the invention, a chain is a group of atoms which is pendent or in the side with respect to the polymer backbone.

A semicrystalline polymer suitable for the invention may in particular have a melting point above the temperature of the skin.

According to one particular embodiment, a semicrystalline polymer with one or more crystallizable side chain(s) which is suitable for the invention may have a melting point of less than or equal to 80° C., and in particular within a range of from 30° C. to 70° C., in particular from 40° C. to 65° C., more particularly from 42° C. to 60° C., or from 44° C. to 56° C., or even from 44° C. to 54° C., and more particularly less than 50° C.

This melting temperature or point (Mp) can be measured by any known method, and in particular with a differential scanning calorimeter (DSC).

According to one particular embodiment of the invention, the polymer may be chosen from the homopolymers resulting from the polymerization of at least one monomer comprising a crystallizable side chain, chosen from saturated C₁₀ to C₃₀ alkyl (meth)acrylates, which may be represented by the following formula:

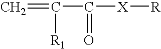

in which R₁ is H or CH₃, R represents a C₁₀ to C₃₀ alkyl group, and X represents O.

According to one particular embodiment of the invention, the polymer is derived from the polymerization of monomers with a crystallizable chain, chosen from saturated C₁₀ to C₃₀ alkyl (meth)acrylates, and more particularly the homopolymers resulting from the polymerization of a monomer with a crystallizable chain, chosen from C₁₄-C₂₄ alkyl acrylates and C₁₄-C₂₄ alkyl methacrylates.

By way of particular example of semicrystalline polymers that can be used in a composition according to the invention, mention may be made of the Intelimer TM products from the company Landec described in the “Intelimer TM polymers” brochure, Landec IP22. These polymers are in solid format at ambient temperature. They bear crystallizable side chains and correspond to saturated C₁₄-C₂₄ alkyl acrylate or methacrylate homopolymers.

As semicrystalline polymers suitable for the compositions according to the invention, mention may also be made of the stearyl acrylate homopolymer, such as the product sold under the name Intelimer® IPA 13-1, from the company Landec or from the company Air Product and Chemicals, or the behenyl acrylate homopolymer (Intelimer IPA-13.6) (INCI name: Poly C 10-30 alkyl acrylate).

The texturing of the composition may be advantageously adjusted according to the nature of the polymers and the respective concentrations thereof.

In particular, the amount of semicrystalline polymer(s) is adjusted so as to provide the smoothing expected on application of the composition under consideration to the skin.

According to one advantageous embodiment of the invention, a composition of the invention may comprise at least 0.6% by weight, in particular at least 1% by weight of semicrystalline polymer with one or more crystallizable side chain(s), as defined above, relative to the total weight of the composition, better still from 1% to 5%, even better still from 1% to 3% by weight of semicrystalline polymer with one or more crystallizable side chain(s), relative to the total weight of the composition.

Those skilled in the art will know how to adjust the presence of the latter in such a way that the required properties of the compositions of the invention are not substantially affected.

According to one particular embodiment of the invention, a composition according to the invention comprises retinol or retinyl palmitate in combination with adenosine and a C₁₀-C₃₀ (meth)acrylate homopolymer, in particular poly(stearyl acrylate).

According to one variant embodiment, the compositions according to the invention may combine, with said semicrystalline polymer with one or more crystallizable side chain(s), at least one secondary polymer, which is in particular useful in terms of texturing properties.

### Additional Polymer

A composition of the invention may also comprise, in addition to the semicrystalline polymer with one or more crystallizable side chain(s) described above, an additional polymer chosen from acrylic polymers, hydrophobically modified polysaccharides, and fatty acid esters of polyols.

Preferably, the additional polymer is hydrophilic.

### Acrylic Polymers

These acrylic polymers may or may not be hydrophilic.

a-Hydrophilic Acrylic Polymers

According to the invention, the term “hydrophilic acrylic polymers” is intended to mean in particular non-amphiphilic and non-hydrophobic acrylic polymers.

Said hydrophilic acrylic polymers according to the invention are either acrylic polymers of polyacrylamidomethylpropanesulphonic acid (AMPS®) or acidic acrylic polymers.

The presence of this hydrophilic acrylic polymer makes it possible in particular to obtain a composition which exhibits good stability properties.

Among the hydrophilic acrylic polymers, mention may in particular be made of the following polymers.

### 1) Acrylic Polymers Containing at Least One Monomer Comprising a Sulphonic Group

According to a first embodiment, the hydrophilic acrylic polymer used according to the invention contains at least one monomer comprising a sulphonic

The polymers containing at least one monomer comprising a sulphonic group that are used in the composition of the invention are advantageously water-soluble or water-dispersible or swellable in water. The polymers used in accordance with the invention are homopolymers obtainable from at least one ethylenically unsaturated monomer comprising a sulphonic group, which may be in free form or in partially or totally neutralized form.

Preferably, the polymers in accordance with the invention may be partially or totally neutralized with an inorganic base (sodium hydroxide, potassium hydroxide, aqueous ammonia) or an organic base such as mono-, di- or triethanolamine, an aminomethylpropanediol, N-methylglucamine, basic amino acids such as arginine and lysine, and mixtures of these compounds. They are generally neutralized. In the present invention, the term “neutralized” is intended to mean polymers which have been totally or near-totally neutralized, in other words neutralized to an extent of at least 90%.

The polymers used in the composition of the invention generally have a number-average molecular weight ranging from 1000 to 20 000 000 g/mol, preferably ranging from 20 000 to 5 000 000, and even more preferably from 100 000 to 1 500 000 g/mol.

These polymers according to the invention may be crosslinked or noncrosslinked.

The monomers comprising a sulphonic group, of the polymer used in the composition of the invention, are chosen in particular from vinylsulphonic acid, styrenesulphonic acid, (meth)acrylamido(C₁-C₂₂)alkylsulphonic acids, N-(C₁-C₂₂)alkyl-(meth)acrylamido-(C₁-C₂₂)alkylsulphonic acids such as undecylacrylamidomethanesulphonic acid, and also their partially or totally neutralized forms, and mixtures thereof.

According to one preferred embodiment of the invention, the monomers comprising a sulphonic group are chosen from (meth)acrylamido(C₁-C₂₂)alkylsulphonic acids such as, for example, acrylamidomethanesulphonic acid, acrylamidoethanesulphonic acid, acrylamidopropanesulphonic acid, 2-acrylamido-2-methylpropanesulphonic acid, 2-methacrylamido-2-methylpropanesulphonic acid, 2-acrylamido-n-butanesulphonic acid, 2-acrylamido-2,4,4-trimethylpentanesulphonic acid, 2-methacrylamidododecylsulphonic acid, 2-acrylamido-2,6-dimethyl-3-heptanesulphonic acid, and also their partially or totally neutralized forms, and mixtures thereof.

More particularly, use is made of 2-acrylamido-2-methylpropanesulphonic acid (AMPS®), and also its partially or totally neutralized forms.

When the polymers are crosslinked, the crosslinking agents may be chosen from the compounds containing olefinic polyunsaturation that are commonly used for the crosslinking of polymers obtained by free-radical polymerization.

As crosslinking agents, mention may, for example, be made of divinylbenzene, diallyl ether, dipropylene glycol diallyl ether, polyglycol diallyl ethers, triethylene glycol divinyl ether, hydroquinone diallyl ether, ethylene glycol di(meth)acrylate or tetraethylene glycol di(meth)acrylate, trimethylolpropane triacrylate, methylenebisacrylamide, methylenebismethacrylamide, triallylamine, triallyl cyanurate, diallyl maleate, tetraallyl-ethylenediamine, tetraallyloxyethane, trimethylolpropane diallyl ether, allyl (meth)acrylate, the allyl ethers of alcohols from the sugar series, or other allyl or vinyl ethers of polyfunctional alcohols, and also allyl esters of derivatives of phosphoric and/or vinylphosphonic acid, or the mixtures of these compounds.

According to one preferred embodiment of the invention, the crosslinking agent is chosen from methylenebisacrylamide, allyl methacrylate or trimethylolpropane triacrylate (TMPTA). The degree of crosslinking ranges in general from 0.01 to 10 mol %, and more particularly from 0.2 to 2 mol %, relative to the polymer.

The homopolymer of monomers comprising a sulphonic group may be crosslinked with one or more crosslinking agents.

These homopolymers are generally crosslinked and neutralized, and they may be obtained according to the preparation process comprising the following steps:
(a) the monomer, such as 2-acrylamido-2-methylpropanesulphonic acid, is dispersed or dissolved in free form in a solution of tert-butanol or of water and tert-butanol;
(b) the solution or dispersion of monomer obtained in (a) is neutralized with one or more organic or inorganic bases, preferably ammonia NH₃, in an amount producing a degree of neutralization of the sulphonic acid functions of the polymer ranging from 90% to 100%;
(c) the crosslinking monomer(s) is (are) added to the solution or dispersion obtained in (b);
(d) a conventional free-radical polymerization is carried out in the presence of free-radical initiators at a temperature ranging from 10 to 150° C.; the polymer precipitating in the solution or dispersion based on tert-butanol.

The preferred AMPS® homopolymers are generally characterized in that they comprise, distributed at random:
a) from 90% to 99.9% by weight of units of general formula (II) below:

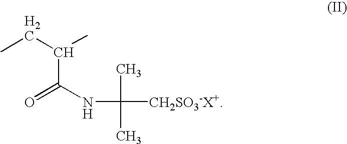

in which X⁺ denotes a proton, an alkali metal cation, an alkaline-earth metal cation or the ammonium ion, no more than 10 mol % of the cations X⁺ being able to be protons H⁺;
b) from 0.01% to 10% by weight of crosslinking units originating from at least one monomer having at least two olefinic double bonds; the proportions by weight being defined relative to the total weight of the polymer.

The homopolymers according to the invention that are more particularly preferred comprise from 98% to 99.5% by weight of units of formula (II) and from 0.2% to 2% by weight of crosslinking units.

As polymers of this type, mention may in particular be made of the crosslinked and neutralized homopolymer of 2-acrylamido-2-methylpropanesulphonic acid sold by the company Clariant under the trade name Hostacerin® AMPS® (CTFA name: ammonium polyacryldimethyltauramide).

### 2) Acrylamide/AMPS® Copolymers

According to another embodiment, the hydrophilic acrylic polymer is a crosslinked anionic copolymer composed of units deriving from the reaction of (i) acrylamide (monomer 1), (ii) 2-actylamido-2-methylpropanesulphonic acid (monomer 2, hereinafter called AMPS® for convenience) and (iii) at least one compound containing olefinic polyunsaturation (monomer 3), here constituting the crosslinking agent.

The crosslinked anionic copolymers used in the context of the present invention are products that are already known per se, and the preparation thereof has been described in particular in patent application EP-A-0 503 853, the content of which is consequently included completely by way of reference in the present description.

The above copolymers may thus be obtained conventionally by the technique known as emulsion polymerization, from the three different comonomers of which they are composed.

The monomers containing olefinic polyunsaturation that are used as crosslinking agents for preparing the copolymers in accordance with the invention are preferably chosen from the group constituted of methylenebisacrylamide, allylsucrose and pentaerythritol. Even more preferably, use is made of methylenebisacrylamide.

Preferably, said compound containing olefinic polyunsaturation is present in the copolymer at a concentration of between 0.06 and 1 millimol per mole of the entirety of the monomer units.

The ratio, expressed in mol %, between the acrylamide and the AMPS® is preferably between 85/15 and 15/85, advantageously between 70/30 and 30/70, even more preferably between 65/35 and 35/65, and even more particularly between 60/40 and 40/60. In addition, the AMPS® is generally at least partially neutralized in the form of a salt, for example with sodium hydroxide, with potassium hydroxide or with a low-molecular-weight amine such as triethanolamine, or mixtures thereof.

A crosslinked copolymer which is particularly preferred in the context of the implementation Of the present invention corresponds to the copolymer as prepared in example 1 of patent application EP-A-0 503 853 mentioned above, which is then in the form of an inverse water-in-oil emulsion. More specifically, this copolymer is composed of 60 mol % of acrylamide and 40 mol % of the sodium salt of AMPS®, and it is crosslinked with methylenebisacrylamide in a proportion of 0.22 millimol per mole of the total mixture of monomers. The final inverse water-in-oil emulsion itself contains, preferably, approximately 40% by weight of crosslinked copolymer as defined above and of the order of 4% by weight of an ethoxylated fatty alcohol having an HLB of approximately 12.5.

As crosslinked copolymer, use is more particularly made, according to the invention, of the products sold under the name Sepigel 305 (CTFA name: polyacrylamide/C13-14 isoparaffin/Laureth 7) or Simulgel 600 (CTFA name: acrylamide/sodium acryloyldimethyltaurate copolymer/isohexadecane/polysorbate 80), sold by the company Seppic.

### 3) Other Hydrophilic Acrylic Polymers

As other hydrophilic acrylic polymers that can be used according to the invention, mention may also be made of:
homopolymers or copolymers of acrylic acid or their salts, such as the products sold under the names Carbopol 934, 940, 954, 981 and 980 by the company Noveon, Synthalen L® from the company 3V, the poly(sodium methacrylate) sold under the name Darvan No. 7® by the company Vanderbilt;
glyceryl acrylate polymers, and in particular the copolymers of glyceryl acrylate and acrylic acid, such as the products sold under the names Lubrajel® MS, Lubrajel® CG,

Lubrajel® DV, Lubrajel® NP, Lubrajel® 011, Lubrajel® Oil BG, Lubrajel® PF, Lubrajel® TW and Lubrajel® WA by the company Guardian Laboratories. Use is preferably made of Lubrajel® MS.
copolymers of acrylic acid salt/vinyl alcohol, such as the product sold under the name Hydragen FN® by Cognis,
and mixtures thereof.
   b-Hydrophic Acrylic Polymers

Such polymers may be derived from the AMPSs described above. These polymers comprise both a hydrophilic part and a hydrophobic part comprising at least one fatty chain. They are therefore amphiphilic polymers.

A fatty chain of such a polymer may contain from 7 to 30 carbon atoms, and in particular from 8 to 22 carbon atoms.

A hydrophobic AMPS® copolymer in accordance with the invention may have a weight-average molecular weight ranging from 50 000 to 10 000 000, in particular from 100 000 to 8 000 000, and more particularly from 100 000 to 7 000 000.

A hydrophobic AMPS® copolymer according to the invention may be crosslinked or non-crosslinked.

Among the crosslinking agents that may be suitable, mention may be made, in a nonlimiting manner, of methylenebisacrylamide, allyl methacrylate or trimethylolpropane triacrylate (TMPTA).

The degree of crosslinking may range from 0.01 to 10 mol %, and in particular from 0.2 to 2 mol %, relative to the polymer.

An amphiphilic AMPS® polymer suitable for the invention may, for example, be chosen from random amphiphilic polymers of AMPS® modified by reaction with an N-mono-alkylamine or a di-N-alkylamine which is C₆-C₂₂, such as those described in patent application WO 00/31154.

These polymers may also contain other ethylenically unsaturated hydrophilic monomers chosen, for example, from acrylic acid, methacrylic acid or their alkyl-substituted derivatives or their esters obtained with monoalkylene glycols or polyalkylene glycols, acrylamide, methaerylamide, vinylpyrrolidone, itaconic acid or maleic acid, or mixtures thereof.

A polymer of the invention may be chosen from amphiphilic polymers of AMPS® and of at least one ethylenically unsaturated monomer comprising at least one hydrophobic part containing from 7 to 30 carbon atoms, and in particular from 8 to 22 carbon atoms, and more particularly from 12 to 20 carbon atoms.

The hydrophobic part may be a saturated or unsaturated, linear alkyl radical (for example, n-octyl, n-decyl, n-hexadecyl, n-dodecyl or oleyl radical), a branched alkyl radical (for example, isostearyl radical) or a cyclic alkyl radical (for example, cyclododecane or adamantane radical).

A polymer suitable for the invention may also contain one or more ethylenically unsaturated hydrophobic monomers comprising, for example:
a fluoro or C₇-C₁₈ alkyl fluoro radical (for example the group of formula —(CH₂)₂—(CF₂)₉—CF₃),
a cholesteryl radical or a cholesterol-derived radical (for example, cholesteryl hexanoate),
an aromatic polycyclic group such as naphthalene or pyrene,
a silicone radical or alkylsilicone radical or else alkylfluorosilicone radical.

Such copolymers are described, for example, in patent application EP-A-0 750 899 and patent U.S. Pat. No. 5,089,578 and in the publications by Yotaro Morishima: “Self-assembling amphiphilic polyelectrolytes and their nanostructures—Chinese Journal of Polymer Science Vol. 18, N(40), (2000): 323-336”; “Micelle formation of random copolymers of sodium 2-(aerylamido)-2-methylpropanesulfonate and a non-ionic surfactant macromonomer in water as studied by fluorescence and dynamic light scattering—Macromolecules 2000, Vol. 33 N(10): 3694-3704”; “Solution properties of micelle networks formed by non-ionic moieties covalently bound to an polyelectrolyte: salt effects on rheological behavior—Langmuir, 2000,Vol. 16 N(12): 5324-5332”; “Stimuli responsive amphiphilic copolymers of sodium 2-(acrylamido)-2-methylpropanesulfonate and associative macromonomers—Polym. Preprint, Div. Polym. Chem. 1999, 40(2): 220-221”. They are also described in patent applications (Clariant): EP 1 069 142,WO 02/44224, WO 02/44225, WO 02/44227, WO 02/44229, WO 02/44230, WO 02/44231, WO 02/44267, WO 02/44268, WO 02/44269, WO 02/44270, WO 02/44271, WO 02/43677, WO 02/43686, WO 02/43687, WO 02/43688, WO 02/43689.

An ethylenically unsaturated hydrophobic monomer suitable for the invention may be chosen from the acrylates or the acrylamides of formula (I) below:

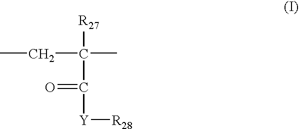

in which:
R₂₇ denotes a hydrogen atom, or a linear or branched C₁-C₆ alkyl radical (preferably methyl),
Y denotes O or NH,
R₂₈ denotes a hydrophobic radical comprising a fatty chain containing from 7 to 30 carbon atoms, in particular from 8 to 22, and more particularly from 12 to 20 carbon atoms.

The hydrophobic radical R₂₈ may in particular be chosen from saturated or unsaturated, C₇-C₂₂ linear alkyl radicals (for example n-octyl, n-decyl, n-hexadecyl, n-dodecyl or oleyl radicals), branched alkyl radicals (for example, isostearyl radicals) or cyclic alkyl radicals (for example, cyclododecane or adamantane radicals); C₇-C₁₈ alkyl perfluoro radicals (for example, the group of formula —(CH₂)₂—(CF₂)₉—CF₃); the cholesteryl radical or a cholesterol ester such as cholesteryl hexanoate; and aromatic polycyclic groups such as naphthalene or pyrene.

Among these radicals, linear and branched alkyl radicals will be more particularly used.

According to one embodiment of the invention, the hydrophobic radical R₂₈ may also comprise at least one alkylene oxide unit, and in particular a polyoxyalkylenated chain.

A polyoxyalkylenated chain may be composed of ethylene oxide units and/or of propylene oxide units, and more particularly may be composed only of ethylene oxide units.

The number of moles of oxyalkylenated units may range generally from 1 to 30 mol, and more particularly from 2 to 25 mol, and even more particularly from 3 to 20 mol.

Among the amphiphilic AMPS® polymers suitable for the invention, mention may be made of:
crosslinked or noncrosslinked, neutralized or non-neutralized copolymers comprising from 15% to 60% by weight of AMPS® units and from 40% to 85% by weight of (C₈-C₁₆)alkyl(meth)acrylarnide units or of (C₈-C₁₆)alkyl(meth)acrylate units, relative to the polymer, such as those described in patent application EP-A-750 899;
terpolymers comprising from 10 to 90 mol % of acrylamide units, from 0.1 to 10 mol % of AMPS® units and from 5 to 80 mol % of n-(C₆-C₁₈)alkylacrylamide units, relative to the polymer, such as those described in patent U.S. Pat. No. 5,089,578.

As amphiphilic polymers suitable for the invention, mention may also be made of the copolymers of totally neutralized AMPS® and of n-dodecyl, n-hexadecyl and/or n-octadecyl methacrylate, and also the non-crosslinked and crosslinked copolymers of AMPS® and of n-dodecylmethacrylamide.

Mention may also be made of the crosslinked or non-crosslinked amphiphilic copolymers comprising, or composed of:
(a) the 2-acrylamido-2-methylpropanesulphonic acid (AMPS®) units of formula (2) below:

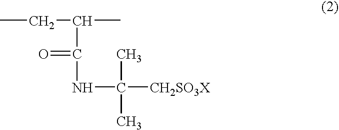

in which X may be a proton, an alkali metal cation, an alkaline-earth metal cation or the ammonium ion;
(b) the units of formula (3) below:

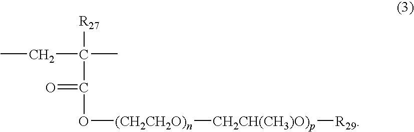

in which:
n and p, independently of one another, denote a number of moles and range from 0 to 30, in particular from 1 to 25 and more particularly from 3 to 20, with the proviso that n+p is less than or equal to 30, in particular less than 25 and more particularly less than 20:
R₂₇ has the same meaning indicated above in formula (1) above, and
R₂₉ denotes a linear or branched alkyl containing m carbon atoms, m ranging from 7 to 22, preferably from 12 to 20.

In formula (2), the cation X may denote more particularly sodium or ammonium.

Among the monomers of formula (3), mention may be made of
esters of (meth)acrylic acid and of a polyoxyethylenated C₁₀-C₁₈ fatty alcohol comprising 8 EO, such as the product Genapol C-080 sold by the company Clariant,
esters of (meth)acrylic acid and of a polyoxyethylenated C₁₁ fatty oxoalcohol comprising 8 EO, such as the product Genapol UD-080 sold by the company Clariant,
esters of (meth)acrylic acid and of a polyoxyethylenated C₁₂-C₁₄ fatty alcohol comprising 7 EO, such as the product Genapol LA-070 sold by the company Clariant,
esters of (meth)acrylic acid and of a polyoxyethylenated C₁₂-C₁₄ fatty alcohol comprising 11 EO, such as the product Genapol LA-I10 sold by the company Clariant,
esters of (meth)acrylic acid and of a polyoxyethylenated C₁₆-C₁₁ fatty alcohol comprising 8 EO. such as the product Gcnapol T-080 sold by the company Clariant,
esters of (meth)acrylic acid and of a polyoxyethylenated C₁₆-C₁₈ fatty alcohol comprising 15 EO, such as the product Genapol T-150 sold by the company Clariant,
esters of (meth)acrylic acid and of a polyoxyethylenated C₁₆-C₁₈ fatty alcohol comprising 11 EO, such as the product Genapol T-110 sold by the company Clariant,
esters of (meth)acrylic acid and of a polyoxyethylenated C₁₆-C₁₈ fatty alcohol comprising 20 EO, such as the product Genapol T-200 sold by the company Clariant,
esters of (meth)acrylic acid and of a polyoxyethylenated C₁₆-C18 fatty alcohol comprising 25 EO, such as the product Genapol T-250 sold by the company Clariant,
esters of (meth)acrylic acid and of a polyoxyethylenated C₁₈-C₂₂ fatty alcohol comprising 25 EO and/or of a polyoxyethylenated C₁₆-C₁₈ fatty isoalcohol comprising 25 EO.

Mention may more particularly be made of:
i. the non-crosslinked polymers for which p=0, n=7 or 25, R₂₇ denotes methyl and R₂₉ represents a mixture of C₁₂-C₁₄ or C₁₆-C₁₈ alkyl, and
ii, the crosslinked polymers for which p=0, n=8 or 25, R₂₇ denotes methyl and R₂₉ represents a mixture of C₁₆-C₁₈ alkyl.

These polymers are described and synthesized in document EP 1 069 142.

These particular amphiphilic polymers may be obtained according to the conventional free-radical polymerization processes in the presence of one or more initiators, such as, for example, azobisisobutyronitrile (AIBN), azobisdimethylvaleronitrile, 2,2-azobis[2-amidinopropane] hydrochloride (ABAH), organic peroxides such as dilauryl peroxide, benzoyl peroxide, tert-butyl hydroperoxide, etc., inorganic peroxide compounds such as potassium persulphate or ammonium persulphate, or H₂O₂ optionally in the presence of reducing agents.

These amphiphilic polymers may be obtained by free-radical polymerization in a tert-butanol medium in which they precipitate.

By using polymerization via precipitation in tert-butanol, it is possible to obtain a particle size distribution for the polymer which is particularly favourable for uses thereof.

The reaction may be carried out at a temperature of between 0 and 150° C., in particular between 10 and 100° C., either at atmospheric pressure or at reduced pressure.

The reaction may also be carried out under an inert atmosphere, and preferably under nitrogen.

A polymer in accordance with the invention may be partially or completely neutralized with an inorganic or organic base such as those mentioned above.

The percentage molar concentration of the units of formula (2) and of the units of formula (3) in an amphiphilic polymer according to the invention may vary according to the desired cosmetic use, to the nature of the emulsion (oil-in-oil or water-in-oil) and to the rheological properties of the desired formulation.

It may range between 0.1 and 99.9 mol %.

The molar proportion of units of formula (3) in an amphiphilic polymer according to the invention may preferably range from 0.1% to 50%, more particularly from 1% to 25%, and even more particularly from 3% to 10%.

The molar proportion of units of formula (3) in an amphiphilic polymer according to the invention may preferably range from 50.1% to 99.9%, more particularly from 60% to 95%, and even more particularly from 65% to 90%.

The distribution of the monomers in the polymers of the invention may, for example, be alternating, block (including multiblock) or any distribution.

By way of indication, and without this being limiting, mention may be made of the following commercial references: Aristoflex® HMS and Aristoflex® HMB sold by Clariant, these two references relating to crosslinked polymers.

Aristoflex® HMS has the name 80/20 AMPS®/ethoxylated (25 EO) cetearyl methacrylate copolymer crosslinked with trimethylolpropane triacrylate (TMPTA), or else the INCI name ammonium acryloyldimethyltaurate/steareth-25 methacrylate crosspolymer.

The INC' name of Aristoflex® HMB is ammonium acryloyldimethyltaurate/beheneth-25 methacrylate crosspolymer.

As hydrophobic AMPS® copolymer, use may also be made of non-crosslinked AMPS® copolymers (Aristoflex® LNC or SNC), which are also effective in terms of emulsion stabilization. On the other hand, the textures then obtained are less original, in particular from the point of view of the water transformation effect.

The INCI name of Aristoflex® LNC is ammonium acryloyldimethyltaurate/laureth-7 methacrylate copolymer.

The INCI name of Aristoflex® SNC is ammonium acryloyldimethyltaurate/steareth-8 methacrylate copolymer.

Among the acrylic polymers that may be combined with a combination in accordance with the invention, mention may also be made of neutralized, crosslinked acrylic homopolymers or copolymers.

c-Neutralized, Crosslinked Acrylic Homopolymers or Copolymers

The main role of this polymer present in the composition of the invention lies in the gelling of the aqueous phase.

All crosslinked acrylic homopolymers or copolymers are suitable for the present invention provided that they are used in an at least partially neutralized form.

By way of these crosslinked acrylic polymers possibly already neutralized before their use, mention is made, for example, of:
Cosmedia SP® or crosslinked poly(sodium acrylate) containing 90% solids and 10% water, Cosmedia SPL® or poly(sodium acrylate) as an inverse emulsion containing approximately 60% solids, an oil (hydrogenated polydecene) and a surfactant (PPG-5 laureth-5), both sold by the company Cognis,
carbopols,
partially neutralized, crosslinked poly(sodium acrylatc)s which are in the form of an inverse emulsion, comprising at least one polar oil, for example that sold under the name Luvigel® EM by the company BASF, and
mixtures thereof.

A crosslinked acrylic acid polymer in accordance with the present invention, not previously neutralized, may be neutralized by any suitable means, and in particular by adding sodium hydroxide. Poly(sodium acrylate(s) are thus obtained. Poly(potassium acrylate)s are also suitable for the present invention.

In reality, the neutralization may be carried out prior to the use in the composition of the invention if the polymer in question is sold in a non-neutralized form. On the other hand, for some of them, the neutralization is inherent in the starting material. This is the case in particular of Luvigel® EM and of the products called Cosmédia® SP and SPL, which is/are already partially neutralized.

The step of neutralization, for example by means of the sodium or potassium counterions, is necessary to confer on the crosslinked acidic polymers their properties of gelling arid therefore of stabilizing the composition. Said crosslinked acrylic polymers are converted to the corresponding acrylate polymers during this neutralization step. The acrylic monomers of the crosslinked acrylic polymer according to the invention may be neutralized to a degree of from 5% to 80%.

According to one particular embodiment of the invention, the crosslinked acrylic polymer in accordance with the invention may comprise ionic monomers. As ionic monomers, use may be made of acrylamide, methacrylamide, vinylpyrrolidone, vinylimidazole, vinylcaprolactam and hydroxyalkyl esters of carboxylic acids, such as hydroxyethyl acrylates. As ionic monomer, mention may in particular be made of unsaturated C₃-C₅ carboxylic acid. However, in the context of the present invention, crosslinked acrylic polymers comprising more than 90% of acrylic acid monomers, or even comprising no nonionic monomer, are preferred.

According to one particular embodiment, the crosslinked acrylic acid homopolymer or copolyrner may be in the form of a water-in-oil emulsion, termed inverse emulsion. This inverse emulsion may, for example, be obtained by inverse emulsion polymerization.

According to one particular embodiment of the invention, the gelling polymer used is a partially neutralized, crosslinked poly(sodium acrylate) in the form of an inverse emulsion comprising at least one polar oil. Among the oils, mention may be made of fatty acid esters. Examples of these fatty acid esters are isopropyl esters of fatty acids, such as isopropyl palmitate or isopropyl myristate, or polyglycerides of fatty acids, in particular of mixtures of fatty acids comprising at least 50% by weight of capric and/or caprylic acids. Such water-in-oil emulsions are described in document U.S. Pat. No. 6,197,283, which is incorporated into the present application by way of reference.

According to this embodiment, the oily phase may be constituted of one or more fatty acid esters, of one or more fatty acid polyglycerides based on a mixture of polyglycerides, which contains diglycerides and triglycerides, with mixtures of fatty acids, which contain caprylic acid and/or capric acid, preferably in a proportion of at least 50% by weight relative to the total weight of fatty acids.

According to one embodiment of the invention, the oil content of the inverse emulsion is between 15% and 70% by weight, in particular between 20% and 35% by weight, relative to the total weight of the inverse emulsion.

In this respect, mention is in particular made of Luvigel® EM, the oily phase of which comprises 26% of oil phase constituted of C₈-C₁₀ triglycerides, i.e. the fatty acids of which arc a mixture of capric acid and caprylic acid.

Moreover, the water-in-oil emulsion may contain from 0.25% to 7% by weight, preferably 0.5% to 5% by weight, of a surfactant.

The at least partially neutralized, crosslinked acrylic polymer may he present in the inverse emulsion in a content ranging from 20% to 70% by weight, in particular from 20% to 65% by weight, for example from 20% to 62% by weight, relative to the total weight of the inverse emulsion.

In particular, according to one embodiment, the crosslinked acrylic polymer may be present in the inverse emulsion in a content ranging from 20% to 30% by weight relative to the total weight of the inverse emulsion. According to yet another embodiment, the crosslinked acrylic polymer may be present in the inverse emulsion in a content ranging from 50% to 62% by weight relative to the total weight of the composition.

The polymers in accordance with the invention may be constituted
a) of 35% to 100% by weight of ionic monomers, the ionic monomers being neutralized to a degree of 5-80%,
b) of 0 to 65% by weight of non-ionic monomers,
c) of 0.3 mol % to 1 mol %, relative to a) and b), of at least one monomer which is at least bifunctional.

In the water-in-oil formulation of such a polymer, the oily phase may then be constituted of one or more fatty acid esters as described above.

The crosslinking of the acrylic acids may be obtained according to any method known to those skilled in the art, in particular according to the description of document U.S. Pat. No. 6,197,283 or according to the description of document U.S. Pat. No. 6,444,785, which mentions the crosslinking agents that can be used.

Among these, mention is made of compounds comprising an unsaturation which are soluble in water or in oil. Such crosslinking agents are in particular methylenebisacrylamide, divinylpyrrolidone, alkyl (meth)acrylate, triallylamine, ethylene glycol diacrylates (up to 50 EO), (meth)acrylic esters with di- or polyhydric alcohols, such as trimethylolpropane triacrylato or pentaerythrityl tetraacrylate.

According to one embodiment, the crosslinking agent is water-soluble.

According to another embodiment, the crosslinking agent is triallylamine.

The preparation of W/O emulsions comprising a polymer in accordance with the present invention may be carried out according to the teaching of document U.S. Pat. No. 6,444,785, incorporated herein by way of reference. The objective of this method is to reduce the content of remaining monomers by post-treatment with a redox initiator system. According to this method, the post-treatment of the W/O emulsion is carried out by addition of a redox initiator system which essentially comprises:
a) 0.001% to 5% by weight, relative to the total amount of monomers used for the preparation of the polymer,
a1) of an oxidizing agent R¹OOH,
in which R¹ denotes hydrogen, a C₁ to C₈ alkyl group or a C₆ to C₁₂ aryl group, and/or
a2) of a compound which releases hydrogen peroxide in an aqueous medium, and
b) 0.005% to 5% by weight, relative to the total amount of monomers used for the preparation of the polymer,
b1) of an α-hydroxycarbonyl compound

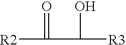

in which the groups have, independently of one another, the following meaning:
R2 is hydrogen, or a C₁-C₁₂ alkyl group, which optionally contains functional groups and/or may comprise olefinic unsaturations,
R3 is hydrogen, OH, or a C₁-C₁₂ alkyl group, which optionally contains functional groups and/or may comprise olefinic unsaturations,
and whereas R2 and R3 may form a cyclic structure, which may contain a heteroatom and/or of functional groups, and/or may comprise olefinic unsaturations,
   and/or
b2) of a compound which releases such an α-hydroxycarbonyl compound in an aqueous medium, and
c) catalytic amounts of a plurivalent metal ion which may have several valency states.

### Hydrophobically Modified Polysaccharides

According to the invention, the term “hydrophobically modified polysaccharide” is intended to mean in particular a polysaccharide modified with hydrophobic chains, in particular modified by grafting of hydrophobic chains onto the hydrophilic backbone of said polysaccharide.

According to the invention, the term “polysaccharide” is intended to mean in particular inulins, celluloses and derivatives thereof (methylcelluloses, hydroxyalkyl-celluloses, hydroxyethylcelluloses, hydroxypropylcelluloses, ethylhydroxyethylcelluloses, carboxymethylcelluloses), starches and agars, and mixtures thereof.

Preferably, inulins, celluloses and derivatives thereof, and mixtures thereof will be used. A composition of the invention may thus comprise a hydrophobically modified polysaccharide chosen from inulins, celluloses and derivatives thereof, starches and agars, and mixtures thereof.

### Hydrophobically Modified Inulins

According to a first embodiment, the polysaccharide used in the present invention is chosen from fructans, in particular inulins.

Fructans or fructosans are oligosaccharides or polysaccharides comprising a series of anhydrofructose units optionally combined with one or more saccharide residues other than fructose. The fructans may be linear or branched. The fructans may be products obtained directly from a plant or microbial source or else products of which the chain length has been modified (increased or reduced) by fractionation, synthesis or hydrolysis, in particular of the enzymatic type. The fructans generally have a degree of polymerization of 2 to approximately 1000, and preferably of 2 to approximately 60.

Three groups of fructans can be distinguished. The first group corresponds to products of which the fructose units are mostly linked by β-2-1 linkages. They are fructans that are essentially linear, such as inulins.

The second group also corresponds to linear fructoses, but the fructose units are essentially linked by β-2-6 linkages. These products are levans.

The third group corresponds to mixed fructans, i.e. fructans having β-2-6 ad β-2-1 sequences. They are fructans that are essentially branched, such as graminans.

The fructans used in the compositions according to the invention are inulins. The inulin may be obtained, for example, from chicory, from dahlia or from Jerusalem artichoke. Preferably, the inulin used in the composition according to the invention is obtained, for example, from chicory.

The polysaccharides, in particular the inulins, used in the compositions according to the invention are hydrophobically modified. In particular, they are obtained by grafting hydrophobic chains onto the hydrophilic backbone of the fructan.

The hydrophobic chains that can be grafted onto the main chain of the fructan may in particular be saturated or unsaturated, linear or branched hydrocarbon-based chains containing from 1 to 50 carbon atoms, such as alkyl, arylalkyl, alkylaryl or alkylene groups; cycloaliphatic divalent groups or organopolysiloxane chains. These hydrocarbon-based or organopolysiloxane chains may in particular comprise one or more ester, amide, urethane, carbamate, thiocarbamate, urea, thiourea and/or sulphonamide functions, such as, in particular, methylenedicyclohexyl and isophorone; or aromatic divalent groups such as phenylene.

A composition of the invention may thus comprise, as hydrophobically modified polysaccharide, an inulin modified with saturated or unsaturated, linear or branched hydrophobic chains containing from 1 to 50 carbon atoms, such as alkyl, arylalkyl, alkylaryl or alkylene groups; cycloaliphatic divalent groups or organopolysiloxane chains comprising one or more ester, amide, urethane, carbamate, thiocarbamate, urea, thiourea and/or sulphonamide functions, such as, in particular, methylenedicyclohexyl and isophorone; or aromatic divalent groups such as phenylene.

In particular, the inulin is obtained from chicory.

In particular, the polysaccharide, in particular the inulin, has a degree of polymerization of from 2 to approximately 1000, and preferably from 2 to approximately 60, and a degree of substitution of less than 2 on the base of a fructose unit.

According to one preferred embodiment, the hydrophobic chains have at least one alkyl carbamate group of formula R—NH—CO— in which R is an alkyl group containing from 1 to 22 carbon atoms.

According to a more preferred embodiment, the hydrophobic chains are lauryl carbamate groups.

In particular, by way of nonlimiting illustration of the hydrophobically modified inulins that may be used in the compositions according to the invention, mention may be made of stearoyl inulin, such as those sold under the names Lifidrem INST by the company Engelhard and Rheopearl INS by the company Ciba; palmitoyl inulin; undecylenoyl inulin, such as those sold under the names Lifidrem INUK and Lifidrem INUM by the company Engelhard; and inulin lauryl carbamate, such as the product sold under the name Inutec SP1 by the company Orafti.

In particular, the hydrophobically modified polysaccharide is a lauryl carbamate-grafted inulin, especially derived from the reaction of lauryl isocyanate with an inulin, in particular derived from chicory. By way of example of these compounds, mention may in particular be made of the product sold under the name of Inutec SP1 by the company Orafti.

### Hydrophobically Modified Cellulose Derivatives

According to another embodiment of the invention, the hydrophobically modified polysaccharide is a (C₁-C₃) hydroxyalkyl cellulose modified with hydrophobic chains, in particular hydrophobic groups containing from 8 to 30 carbon atoms.

The hydrophobically modified cellulose derivatives according to the invention are substituted with one or more aliphatic or aromatic, saturated or unsaturated, linear, branched or cyclic C₈-C₃₀ hydrocarbon-based chain(s).

According to one embodiment, the hydrophobic substituent(s) used is (are) C₈-C₃₀, preferably C₁₀-C₂₂, alkyl, atylalkyl or alkylaryl groups.

Preferably, the hydrophobic substituent(s) according to the present invention is (are) C₁₀-C₂₂, preferably C₁₆-C₂₀, saturated alkyl chains, such as cetyl (C₁₆), stearyl (C₁₈) or behenyl (C₂₀) groups.

According to one preferred embodiment, the hydrophobic substituent(s) according to the present invention is (are) a cetyl group or cetyl groups.

These cellulose derivatives comprising one or more hydrophobic substituent(s) according to the invention have a viscosity preferably between 100 and 100 000 mPa·s, and preferably between 200 and 20 000 mPa·s, measured at 25° C. in a solution containing 1% by weight of polymer in water, this viscosity being determined conventionally using a Brookfield LVT viscometer at 6 rpm with the No. 3 spindle.

Among the cellulose derivatives comprising one or more hydrophobic substituent(s) that can be used in the compositions of the invention, mention may preferably be made of the cetyl hydroxyethylcelluloses sold under the names Natrosol Plus Grade 330 CS and Polysurf 67 CS (INCE: Cetyl Hydroxyethylcellulose) by the company Aqualon/Hercules.

### Fatty Acid Esters of Polyols

According to the invention, the term “fatty acid esters of polyols” is intended to mean esters of a fatty acid (or polymers of a fatty acid) and of a polyol in which the fatty acid comprises a C₆-C₂₂, preferably C₁₆-C₂₀, alkyl chain and the polyol is chosen from glycerol, a polyglycerol and sorbitan, and mixtures thereof. The fatty acid may also be in a polymeric form, as is the case of polyhydroxystearic acid (polymer of 12-hydroxystearic acid).

According to one particular embodiment, the fatty acid ester of a polyol is a C₁₆-C₂₀ fatty acid ester of glycerol and/or of sorbitan, and mixtures thereof.

As examples of fatty acids comprising a C₁₆-C₂₀ linear or branched chain, mention may be made of stearic acid, isostearic acid, lauric acid, myrstic acid and palmitic acid. As an example of a C₁₆-C₂₀ fatty acid polymer, mention may be made of poly(12-hydroxystearic acid).

Preferably, stearic acid, isostearic acid, poly(12-hydroxystcaric acid) and mixtures thereof will be used.

The term “polyglycerols” is intended to mean compounds of formula:

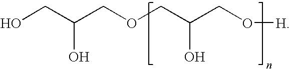

in which the degree of condensation n ranges from 1 to 11, preferably from 2 to 6, and even more preferably from 3 to 6.

According to one particular embodiment, the fatty acid ester of a polyol contains 2 to 10 mol (units) of polyols, preferably 2 to 4 mol of polyols, in particular 2 to 4 units of glycerol or a mixture of polyglycerols (glycerol, di-, tri-, tetra-, penta-oligoglycerols).

Even more preferably, the fatty acid ester of a polyol contains 4 mol (or units) of polyol, in particular 4 mol (or units) of glycerol.

According to one preferred embodiment, said fatty acid ester of a polyol is, in addition, an ester of a fatty acid, of a dicarboxylic acid containing from 2 to 16 carbon atoms, preferably from 8 to 14 carbon atoms, such as azelaic acid, sebacic acid or dodecanedioic acid, and preferably sebacic acid (C₁₀), and of a polyol.

By way of examples of fatty acid esters of a polyol that can be used in the composition of the invention, mention may be made of isostearic acid esters of polyols and mixtures thereof, in particular isostearic acid esters of glycerol and/or of sorbitan, for instance the polyglycerolated (4 mol) isostearate (INCL name: polyglyceryl-4 isostearate) sold under the name Isolan G134® by the company Goldschmidt, the polyglycerolated (3 mol) diisostearate sold under the name Lameform TGI® by the company Cognis; the polyglycerolated (2 mol) distearate sold under the name Emalex PGSA® by the company Nihon emulsion; the polyglycerolated (10 mol) monoisostearate sold under the name Nikkol decaglyn 1-IS by the company Nihon Surfactant (INCL name: polyglyceryl-10 isostearate);

the polyglyceryl-4 diisostearate polyhydroxystearate sebacate sold under the name Isolan GPS by Goldschmidt; the mixture of sorbitan isostearate and glyceryl isostearate, such as the product sold under the name Arlacel 986 by the company ICI, the mixture of sorbitan isostearate and polyglyceryl isostearate (3 mol) sold under the name Arlacel 1690 by the company Uniqema, and mixtures thereof.

As fatty acid esters of polyglycerol that are preferred according to the invention, mention may in particular be made of: the polyglycerolated (4 mol) isostearate (INCI name: polyglyceryl-4 isostearate) sold under the name Isolan G134® by Goldschmidt, the polyglyceryl-4 diisostearate polyhydroxystearate sebacate sold under the name Isolan GPS® by Goldschmidt, the mixture of sorbitan isosteraate and polyglyceryl isostearate (3 mol) sold under the name Arlacel 1690® by the company Uniqema, and mixtures thereof.

According to one preferred embodiment of the invention, the fatty acid ester of a polyol according to the invention is an ester of poly(12-hydroxystearic acid) and of dicarboxylic acids obtained by esterification of a mixture of polyglycerol with (i) a polyhydroxystearic acid, with from 1 to 10, preferably from 2 to 8, even more preferably from 2 to 5, units of polyglycerol (preferably 4 units); (ii) linear or branched, aliphatic dicarboxylic acids containing from 2 to 16 carbon atoms, preferably from 4 to 14 carbon atoms (preferably sebacic acid); and (iii) linear or branched, saturated or unsaturated fatty acids containing from 6 to 22 carbon atoms, preferably from 16 to 20 carbon atoms (preferably isostearic acid). Advantageously, the degree of esterification of the mixture of polyglycerol is between 20% and 40%, preferably between 40% and 70%.

Such poly(12-hydroxystearic acid) esters of polyglycerol are described in application US 2005/0031580.

According to one preferred embodiment, the fatty acid ester of a polyol is an ester of poly(12-hydroxystearic acid) and of dicarboxylic acids obtained by esterification of a mixture of polyglycerol with (i) a polyhydroxystearic acid, with from 2 to 5 units of polyglycerol; (ii) linear or branched, aliphatic dicarboxylic acids containing 4 to 14 carbon atoms and (iii) linear or branched, saturated or unsaturated fatty acids containing from 16 to 20 carbon atoms.

Preferably, the fatty acid ester of a polyol is an ester of poly(hydroxystearic acid) and of dicarboxylic acids obtained by esterification of a mixture of polyglycerol with (i) a poly(hydroxystearic acid). with 2 to 5 units of polyglycerol (preferably 4 units); (ii) linear or branched, aliphatic dicarboxylic acids containing 4 to 14 carbon atoms (preferably sebacic acid) and (iii) linear or branched, saturated or unsaturated fatty acids containing from 16 to 20 carbon atoms (preferably isostearic acid).

As a preferred example of a poly(hydroxystearic acid) ester of polyglycerol, mention may be made of polyglyceryl-4 diisostearate polyhydroxystearate sebacate of formula:

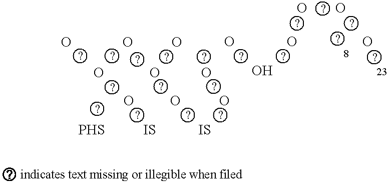

where PHS denotes poly(hydroxystearic acid) and IS denotes isostearic acid.

Such a compound is prepared according to application US 2005/0031580 and sold under the name Isolan GPS® by the company Goldschmidt (Degussa).

According to one particular embodiment of the invention, a composition according to the invention is devoid of 2-acrylamido-2-methylpropanesulphonic acid polymer comprising at least one hydrophobic unit.

### Supplementary Cosmetic Active Agents

According to one advantageous embodiment, the combination according to the invention may be combined with one or more supplementary cosmetic active agents.

These active agents may be chosen from antiwrinkle agents other than adenosine and retinol, UV screening agents, vitamins, in particular B3, B8, B12 and B9, moisturizers, desquamating agents, anti-ageing active agents, agents for improving barrier function, depigmenting agents, antioxidants, dermodecontracting agents or dermorelaxing agents, anti-glycation agents, agents for stimulating the synthesis of dermal and/or epidermal macromolecules and/or for preventing their degradation, agents for stimulating fibroblast or keratinocyte proliferation and/or keratinocyte differentiation, agents for promoting maturation of the horny envelope, NO-synthase inhibitors, peripheral benzodiazepine receptor (PBR) antagonists, agents for increasing the activity of the sebaceous gland, agents for stimulating the energy in the metabolism of cells, and calmatives.

The composition according to the invention may also contain active agents for the care or treatment of greasy skin.

According to one particular variant of the invention, the combination according to the invention is used together with at least one active agent chosen from antiwrinkle active agents other than adenosine, retinol and derivatives thereof, UV screening agents, desquamating agents, antioxidants, agents for stimulating the synthesis of dermal and/or epidermal macromolecules, dermodecontracting agents, and mixtures thereof.

These supplementary active agents may be present in the composition in a content ranging from 0.001% to 20% by weight, preferably from 0.01% to 10% by weight, and more preferably from 0.01% to 5% by weight, relative to the total weight of the composition.

By way of illustration of the anti-ageing active agents, mention may in particular be made of Pro-Xylane or Vigna aconitifolia seed extracts such as those sold by the company Cognis under the references Vitoptine LS9529 and Vit-A-Like LS9737.

### Other Antiwrinkle Agents

Examples of additional antiwrinkle active agents that can be used according to the invention are: ascorbic acid and derivatives thereof, such as magnesium ascorbyl phosphate and ascorbyl glucoside; tocopherol and derivatives thereof, such as tocopheryl acetate; nicotinic acid and precursors thereof, such as nicotinamide; ubiquinone; glutathione and precursors thereof, such as L-2-oxothiazolidine-4-carboxylic acid; C-glycoside compounds and derivatives thereof, as described in particular hereinafter: extracts of plants, and in particular extracts of sea fennel and of olive leaf., and also plant proteins and hydrolysates thereof, such as rice or soybean protein hydrolysates; algal extracts and in particular of laminaria; bacterial extracts; sapogenins, such as diosgenin and extracts of Dioscorea plants, in particular of wild yam, containing them; α-hydroxy acids; β-hydroxy acids, such as salicylic acid and 5-n-octanoylsalicylic acid; oligopeptides and pseudodipeptides and acyl derivatives thereof, in particular {2-[acetyl-(3-trifluoromethylphenyl)amino]-3-methyl-butyrylamino}acetic acid and the lipopeptides sold by the company Sederrna under the trade names Matrixyl 500 and Matrixyl 3000; lycopene; manganese salts and magnesium salts, in particular manganese and magnesium gluconates; and mixtures thereof.

A composition of the invention may in particular use sodium hyaluronate as anti-ageing agent.

The additional antiwrinkle active agent may represent at least 0.05%, preferably at least 0.5%, and more preferably at least 1% by weight, relative to the total weight of the composition.

### Moisturizers

As humectants or moisturizers, mention may in particular be made of urea and derivatives thereof, especially Hydrovance® sold by National Starch, monosaccharides such as mannose, hyaluronic acid, AHAs, BHAs, acrylic acid homopolymers, for instance Lipidure-HM® from NOF Corporation, beta-glucan and in particular sodium carboxymethyl beta-glucan from Mibelle-AG-Biochemistry; a polyoxybutylene polyoxyethylene polyoxypropylene glycerol such as Wilbride S-753L® from NOF Corporation, a rose musk oil sold by Nestlé; spheres of collagen and of chondroitin sulphate of marine origin (atelocollagen) sold by the company Engelhard Lyon under the name Marine Filling Spheres; hyaluronic acid spheres such as those sold by the company Engelhard Lyon.

### UV Screening Agents

By way of nonlimiting illustration of the UV screening agents, mention may be made of the following groups:
anthranilates, in particular menthyl anthranilate; benzophenones, in particular benzophenone-1, benzophenone-3, benzophenone-5, benzophenone-6, benzophenone-8, benzophenone-9, benzophenone-12, and preferably benzophenone-2 (oxybenzone), or benzophenone-4 (Uvinul MS40® available from BASF); benzylidenecamphors, in particular 3-benzylidenecamphor, benzylidenecamphorsulphonic acid, camphor benzalkonium methosulphate, polyactylamidomethylbenzylidenecamphor, terephthalylidenedicamphor-sulphonic acid, and preferably 4-methylbenzylidenecamphor (Eusolex 6300® available from Merck); benzimidazoles, and in particular benzimidazilate (Neo Heliopan AP® available from Haarmann and Reimer), or phenylbenzimidazolesulphonic acid (Eusolex 232® available from Merck); benzotriazoles, in particular drometrizole trisiloxane, or methylene[bis(benzo-triazolyl)(tetramethylbutyl)phenol] (Tinosorb M® available from Ciba); cinnamates, in particular cinoxate, DEA methoxycinnamate, diisopropyl methylcinnamate, glycerylethyl-hexanoate dimethoxycinnamate, isopropyl methoxycinnamate, isoamyl cinnamate, and preferably ethocrylene (Uvinul N35® available from BASF), octyl methoxycinnamate (Parsol MCX® available from Hoffmann La Roche) or octocrylene (Uvinul 539® available from BASF); dibenz oylmethanes, in particular butyl methoxydibenzoylmethane (Parsol 1789®); imidazolines, in particular ethylhexyl dimethoxybenzylidene dioxoimidazoline; PABAs, in particular ethyldihydroxypropyl PABA, ethylhexyldimethyl PABA, glyceryl PABA, PABA, PEG-25 PABA, and preferably diethylhexylbutamidotriazone (Uvasorb HEB® available from 3V Sigma), ethylhexyltriazone (Uvinul T150® available from BASF), or ethyl PABA (benzocaine); Mexoryl®; salicylates, in particular dipropylene glycol salicylate, ethylhexyl salicylate, homosalate, or TEA salicylate; triazines, in particular anisotriazine (Tinosorb S® available from Ciba); drometrizole trisiloxane, zinc oxide, titanium dioxide, zinc oxide, iron oxide, zirconium oxide, and cerium oxide, which may be coated or uncoated.

Preferably, cinnamates and salicylates, and mixtures thereof, are used.

The amount of screening agents depends on the desired final use. It may range, for example, from 1% to 30% by weight, and better still from 2% to 21% by weight, relative to the total weight of the composition containing it.

### Desquamating Agents

As preferred desquamating agents, mention will be made of beta-hydroxy acids, in partictilar salicylic acids and derivatives thereof other than 5-n-octanoylsalicylic acid; urea; glycolic acid, citric acid, lactic acid, tartaric acid, malic acid or mandelic acid; 4-(2-hydroxyethyppiperazine-1-propanesulphonic acid (HEPES); extract of Saphora japonica; honey; N-acetylglucosamine; sodium methylglycine diacetate, alpha-hydroxy acids (ANAs), beta-hydroxy acids (BHAs), and mixtures thereof.

Even more preferably, a desquamating agent such as 4-(2-hydroxy-ethyppiperazine-1-propanesulphonic acid (HEPES) will be used with the combination of the invention.

Thus, a composition in accordance with the invention may comprise a desquamating agent in a content ranging from 0.001% to 10% by weight, preferably from 0.01% to 5% by weight, relative to the total weight of the composition.

### Depigmenting Agents

As depigmenting agents, mention may be made of ceramides, vitamin C and derivatives thereof, in particular vitamin CG, CP and 3-O ethyl vitamin C, alpha- and beta-arbutin, ferulic acid, kojic acid, resorcinol and derivatives thereof, calcium D-pantetheine sulphonate, lipoic acid, ellagic acid, vitamin B3, phenylethyl resorcinol, for instance Symwhite 377® from the company Symrise, a kiwi fruit (Actinidia chinensis) juice sold by Gattefosse, an extract of Paeonia suffructicosa root, such as the product sold by the company Ichimaru Pharcos under the name Botanpi Liquid B®, an extract of brown sugar (Saccharum officinarum), such as the extract of molasses sold by the company Taiyo Kagaku under the name Molasses Liquid, a mixture of undecylenic acid and undecylenoyl phenyl alanine, such as Sepiwhite MSH® from Seppic.

### Antioxidants

As preferred antioxidant, mention may more particularly be made of tocopherol and esters thereof, in particular tocopheryl acetate; EDTA, ascorbic acid and derivatives thereof, in particular magnesium ascorbyl phosphate and ascorbyl glucoside; chelating agents, such as BHT, BHA, N,N′-bis(3,4,5-trimethoxybenzyl)ethylenediamine and its salts, and mixtures thereof.

Tocopherol and esters thereof will more preferably be used.

Thus, a composition in accordance with the invention may comprise an antioxidant in a content ranging from 0.001% to 10%, and preferably from 0.01% to 5% by weight, relative to the total weight of the composition.

### Dermorelaxing or Dermodecontracting Agents

As preferred dermorelaxing agents, mention may more particularly be made of manganese gluconate, wild yam, sea fennel, glycine and alverin.

A composition in accordance with the invention may comprise a dermorelaxing agent in a content ranging from 0.0001% to 5%, and preferably from 0.001% to 3% by weight, relative to the total weight of the composition.

Active Agents for Stimulating the Synthesis of Dermal and/or Epidermal Macromolecules and/or for Preventing their Degradation

As preferred active agents for stimulating the synthesis of dermal and/or epidermal macromolecules and/or for preventing their degradation, mention may be made of: peptides extracted from plants, such as the soybean hydrolysate sold by the company BASF Beauty Care Solutions under the trade name Phytokine®, the malt extract as sold under the name Collalift® by the company Engelhard Lyon; rice peptides such as Nutripeptide® from

Silab, or else an extract of rice peptides, such as Colhibin® from Pentapharm, methylsilanol mannuronate such as Algisium C® sold by Exsymol; an extract of Vaccinium myrtillus, such as those described in application FR-A-2 814 950; the extract of lupin sold by the company Silab under the trade name Structurine®, and mixtures thereof.

A composition in accordance with the present invention may comprise an active agent for stimulating the synthesis of macromolecules in a content ranging from 0.001% to 10%, and preferably from 0.01% to 5% by weight, relative to the total weight of the composition.

The composition according to the invention may also contain Vichy water, rich in minerals, for its fortifying, free-radical-scavenging and soothing action.

### Physiologically Acceptable Medium

A composition containing a combination according to the invention may be intended for cosmetic or pharmaceutical application, particularly dermatological application. Preferably, this composition according to the invention is intended for cosmetic application. it is formulated in a physiologically acceptable medium.

The physiologically acceptable medium is preferably a cosmetically or dermatologically acceptable medium, i.e. which has no unpleasant odour, colour or appearance, and which does not generate any tingling, tautness or redness that is unacceptable for the user.

The term “physiologically acceptable medium” is understood to mean a medium compatible with the keratin materials of human beings, in the case in point the skin.

The composition according to the invention may be in any of the galenical forms normally used in the cosmetics and dermatological fields.

It may in particular be in the form of an aqueous or aqueous-alcoholic, optionally gelled, solution, a dispersion of the lotion type which is optionally two-phase, an oil-in-water or water-in-oil or multiple emulsion, an aqueous gel, a dispersion of oils; in an aqueous phase, in particular by means of spherules, it being possible for the spherules to be polymeric particles or better still lipid vesicles of ionic and/or nonionic type, or else in the form of a powder, a serum, a paste or a flexible stick.

Thus, the composition may comprise all the constituents normally used in the application envisaged.

Mention may in particular be made of water, solvents, oils of mineral, animal and/or plant origin, in particular as described hereinafter, waxes as described hereinafter, in particular pigments, fillers, surfactants, gelling agents and preservatives.

A composition of the invention may advantageously have a firm and compact feel when taken up. It may be thick on application and subsequently transform, melt and release freshness.

Of course, those skilled in the art will take care to select this or these optional additional compound(s), and/or the amount thereof, in such a way that the advantageous properties of the compounds according to the invention are not, or not substantially, impaired by the addition envisaged.

The compositions according to the invention advantageously contain at least one liquid fatty phase.

Advantageously, the compositions according to the invention may be in the form of emulsions.

The compositions according to the invention may advantageously be in the form of an emulsion obtained by dispersion of an aqueous phase in a fatty phase (W/O) or of a fatty phase in an aqueous phase (O/W), of liquid or semi-liquid consistency of the milk type, or of soft, semi-solid or solid consistency of the cream or gel type, or else of a multiple emulsion (W/O/W or O/W/O). These compositions are prepared according to the usual methods.

The compositions of this type may be in the form of a care or makeup product for the face and/or the body, and be packaged, for example, in the form of a cream in a pot or of a fluid in a tube or in a pump-dispenser bottle.

The emulsions generally contain at least one emulsifier chosen from amphoteric, anionic, cationic or nonionic emulsifiers, used alone or as a mixture. The emulsifiers are chosen appropriately according to the emulsion to he obtained (W/O or O/W). The emulsifiers are generally present in the composition in a proportion that may range, for example, from 0.3% to 30% by weight, preferably from 0.5% to 20% by weight, relative to the total weight of the composition.

For the O/W emulsions, emulsifiers that may be mentioned include, for example, nonionic surfactants, and in particular esters of polyols and of fatty acids with a saturated or unsaturated chain containing, for example, from 8 to 24 carbon atoms and better still from 12 to 22 carbon atoms, and the oxyalkylenated derivatives thereof, i.e. derivatives comprising oxyethylenated and/or oxypropylenated units, such as the glyceryl esters of C₈-C₂₄ fatty acids, and the oxyalkylenated derivatives thereof; the polyethylene glycol esters of C₈-C₂₄ fatty acids, and the oxyalkylenated esters thereof; the sorbitol eseters of C₈-C₂₄ fatty acids, and the oxyalkylenated derivatives thereof; the sugar (sucrose, glucose, alkylglucose) esters of C₈-C₂₄ fatty acids, and the oxyalkylenated derivatives thereof; fatty alcohol ethers; the sugar ethers of C₈-C₂₄ fatty alcohols, and mixtures thereof.

As examples of oils that can be used in the composition according to the invention, mention may be made of:
hydrocarbon-based oils of animal origin, such as perhydrosqualene,
hydrocarbon-based oils of plant origin, such as liquid triglycerides of fatty acids containing from 4 to 10 carbon atoms, for instance triglycerides of heptanoic acid or of octanoic acid, or else, for example, sunflower oil, corn oil, soybean oil, marrow oil, grapeseed oil, sesame oil, hazelnut oil, apricot oil, macadamia oil, arara oil, sunflower oil, castor oil, avocado oil, caprylic/capric acid triglycerides such as those sold by the company Stearineries Dubois or those sold under the names Miglyol 810, 812 and 818 by the company Dynamit Nobel, jojoba oil, shea butter oil,
synthetic esters and ethers, in particular of fatty acids, such as the oils of formulae R₁COOR₂ and R₁OR₂ in which R₁ represents the residue of a fatty acid containing from 8 to 29 carbon atoms, and R₂ represents a branched or unbranched hydrocarbon-based chain containing from 3 to 30 carbon atoms, for instance purcellin oil, isononyl isononanoate, isopropyl myristate, 2-ethylhexyl palmitate, 2-octyldodecyl stearate, 2-octyldodecyl erucate, isostearyl isostearate; hydroxylated esters such as isostearyl lactate, octyl hydroxystearate, octyldodecyl hydroxystearate, diisostearyl malate, triisocetyl citrate, heptanoates, octanoates and decanoates of fatty alcohols; polyol esters, for instance propylene glycol dioctanoate, neopentyl glycol diheptanoatc and diethylene glycol diisononanoate; and pentaerythritol esters such as pentaerythrityl tetraisostearate,
linear or branched hydrocarbons of mineral or synthetic origin, such as volatile or non-volatile liquid paraffins, and derivatives thereof, isohexadecane, isododecane, petroleum jelly, polydecenes, hydrogenated polyisobutencs such as Parléam® oil,
natural or synthetic essential oils such as, for example, eucalyptus oil, lavandin oil, lavender oil, vetiver oil, Litsea cubeba oil, lemon oil, sandlewood oil, rosemary oil, camomile oil, savory oil, nutmeg oil, cinnamon oil, hyssop oil, caraway oil, orange oil, geraniol oil, cade oil and bergamot oil,
fatty alcohols containing from 8 to 26 carbon atoms, such as cetyl alcohol, stearyl alcohol, and a mixture thereof (cetylstearyl alcohol), octyldodecanol, 2-butyloctanol, 2-hexyldecanol, 2-undecylpentadecanol, oleyl alcohol or linoleyl alcohol,
partially hydrocarbon-based and/or silicone-based fluoro oils such as those described in document JP-A-2-295912,
silicone oils such as volatile or non-volatile polydimethylsiloxanes (PDMSs) containing a linear or cyclic silicone chain, which are liquid or pasty at ambient temperature, in particular cyclopolydimethylsiloxanes (cyclomethicones) such as cyclohexasiloxane and cyclopentasiloxane; polydimethylsiloxanes comprising alkyl, alkoxy or phenyl groups, which are pendent or at the end of a silicone chain, these groups containing from 2 to 24 carbon atoms; phenylsilicones such as phenyl trimethicones, phenyl dimethicones, phenyltrimethyl-siloxydiphenylsiloxanes, diphenyl dimethicones, diphenylmethyldipheny ltrisiloxanes, 2-phenylethyltrimethylsiloxysilicates and polymethylphenylsiloxanes, and
mixtures thereof.

The term “hydrocarbon-based oil” in the list of oils mentioned above is intended to mean any oil predominantly comprising carbon and hydrogen atoms, and optionally ester, ether, fluoro, carboxylic acid and/or alcohol groups.

The other fatty substances that may be present in the oily phase are, for example, waxes and fatty acids containing from 8 to 30 carbon atoms, for instance stearic acid, lauric acid, palmitic acid and oleic acid.

As waxes that may be used according to the invention, mention may be made of waxes of animal origin, such as beeswax, spermaceti, lanolin wax and lanolin derivatives, plant waxes such as carnauba wax, candelilla wax, ouricury wax, japan wax, cocoa butter or cork fibre wax or sugarcane wax, mineral waxes, for example paraffin wax, petroleum jelly wax, lignite wax or microcrystalline waxes or ozokerites, synthetic waxes, among which are polyethylene waxes, polytetrafluoroethylene waxes and waxes obtained by Fisher-Tropsch synthesis, or else silicone waxes, hydrogenated oils that are solid at 25° C., such as hydrogenated castor oil, hydrogenated jojoba oil, hydrogenated palm oil, hydrogenated tallow or hydrogenated coconut oil, and fatty esters that are solid at 25° C., for instance the C₂₀-C₄₀ alkyl stearate sold under the trade name Kester Wax K82H by the company Koster Keunen.

These fatty substances may be chosen in a varied manner by those skilled in the art in order to prepare a composition having the desired properties, for example of consistency and of texture.

The compositions according to the invention may comprise a volatile oil.

For the purpose of the invention, the term “volatile oil” is intended to mean an oil capable of evaporating on contact with the keratin materials in less than one hour, at ambient temperature and atmospheric pressure. The volatile organic solvent(s) and the volatile oils of the invention are volatile organic solvents and volatile cosmetic oils which are liquid at ambient temperature and which have a non-zero vapour pressure, at ambient pressure and atmospheric pressure, ranging in particular from 0.13 Pa to 40 000 Pa (10⁻³ to 300 mm Hg), in particular ranging from 1.3 Pa to 13 000 Pa (0.01 to 100 mm Hg), and more particularly ranging from 1.3 Pa to 1300 Pa (0.01 to 10 mm Hg).

As volatile oils, mention may be made, inter alia, of cyclic or linear silicones containing from 2 to 6 silicon atoms, such as cyclohexasiloxane, dodecamethylpentasiloxane, decarnethyltetrasiloxane, butyltrisiloxane and ethyltrisiloxane. Use may also be made of branched hydrocarbons such as, for example, isododecane and also volatile perflurooalkanes such as dodecafluoropentane and tetradecafluorohexane, sold under the names PF 5050® and

PF 5060® by the company 3M and perfluoromorpholine derivatives, such as the 4-trifluoromethyl perfluoromorpholine sold under the name PF 5052® by the company 3M.

The amount of oily phase present in the compositions according to the invention may range, for example, from 0.01% to 50% by weight, and preferably from 0.1% to 30% by weight, relative to the total weight of the composition.

The composition according to the invention may also contain silicone elastomers, for instance the products sold under the name KSG by the company Shin-Etsu, under the names Trefil, BY29 or EPSX by the company Dow Corning or under the name Gransil by the company Grant Industries.

The compositions according to the invention may also comprise at least one dyestuff chosen, for example, from pigments, pearlescent agents, colouring agents, materials with'an effect, and mixtures thereof.

These dyestuffs may be present in a content ranging from 0.01% to 50% by weight, preferably from 0.01% to 30%, relative to the total weight of the composition.

The compositions according to the invention may also comprise a filler, in particular in a content ranging from 0.01% to 50% by weight, relative to the total weight of the composition, preferably ranging from 0.01% to 30% by weight. These fillers may be inorganic or organic and of any form, platelet-shaped, spherical or oblong, irrespective of the crystallographic form (for example lamellar, cubic, hexagonal, orthorhombic, or amorphous). Mention may be made of silica, talc, mica, kaolin, lauroyllysine, starch, boron nitride, PTFE powders, PMMA powders, methylsilsesquioxane resin powders (such as Tospearl 145A from GE Silicone), hollow hemispherical particles of silicone resin (such as NLK 500, NLK 506 and NLK 510 from Takernoto Oil and Fat), barium sulphate, precipitated calcium carbonate, magnesium carbonate, magnesium hydrogen carbonate, hydroxyapatite, glass or ceramic microcapsules, metal soaps derived from organic carboxylic acids containing from 8 to 22 carbon atoms, preferably from 12 to 18 carbon atoms, for example zinc stearate, magnesium stearate, lithium stearate, zinc laurate or magnesium myristate.

The composition according to the invention may also contain various adjuvants commonly used in the cosmetics field, such as sequestering agents; fragrances; and thickeners and gelling agents. The amounts of these various adjuvants and the nature thereof will be chosen so as not to be detrimental to the properties of the composition.

The examples and figures which follow serve to illustrate the invention without, however, being limiting in nature. The compounds are, depending on the case, cited as chemical names or CTFA names (International Cosmetic Ingredient Dictionary and Handbook).

### Example 1

### Cream in the Form of an Oil/Water Emulsion

This composition is intended in particular for face and neck care, and may be applied daily.

| Aqueous phase A: | |
|---|---|
| Water | QS 100% |
| Glycerol | 5% |
| Adenosine | 0.1% |
| Preservative | 0.4% |
| Ethylenediaminetetraacetic acid, disodium salt | 0.3% |
| Sodium hyaluronate | 0.1% |
| Crosslinked poly(sodium acrylate) | 0.5% |
| Sucrose monodipalmitostearate | 1.5% |
| Crosslinked acrylic acid/alkyl acrylate polymer | 0.2% |
| Fatty phase B: | |
| Shea butter | 2% |
| Fatty substance | 2% |
| UV screening agents | 15% |
| Stearic acid | 1.2% |
| Poly(stearyl acrylate) | 2% |
| Phase C: | |
| Cyclohexasiloxane | 5% |
| Crosslinked polymer of dimethicone and dimethicone/vinyl | 3% |
| dimethicone | |
| Phase D: | |
| Fillers | 1% |
| Phase E: | |
| Retinol* | 0.07% |

*expressed as active material

### Procedure:

1—heat A and B separately at 80° C. until complete homogenization,
2—add A to B using a turbo mixture at around 75° C.,
3—homogenize,
4—cool to 40° C. and add C using a turbomixer,
5—add D and then, at 25° C., add E.

### Example 2

### Cream in the Form of an Oil/Water Emulsion

This composition is intended in particular for face and neck care, and may be applied daily.

| Aqueous phase A: | |
|---|---|
| Water | QS 100% |
| Glycerol | 7% |
| Sodium hyaluronatc | 0.08% |
| Adenosine | 0.1% |
| Preservatives | 0.9% |
| Ethoxylated AMPS ®/stearyl methacrylate copolymer | 0.8% |
| crosslinked with trimethylolpropane triacrylate | |
| Sucrose monodipalmitostearate | 1.5% |
| Acrylates copolymer | 0.2% |
| Crosslinked poly(sodium acrylate) | 0.8% |
| Solvent | 3% |
| Hydrolysed hyaluronic acid | 0.08% |
| Fatty phase B: | |
| Shea butter | 4% |
| Poly(stearyl acrylate) | 2% |
| Stearic acid | 1.5% |
| Bleached beeswax | 1% |
| Emollients | 10% |
| Phase C: | |
| Cyclohexasiloxane | 3.5% |
| Crosslinked polymer of dimethicone and dimethicone/vinyl | 5% |
| dimethicone | |
| Phase D: | |
| Filler | 2% |
| Phase E: | |
| Retinyl palmitate* | 0.165% |
| Phase F: | |
| Vichy water | 3% |

*expressed as active material

### Procedure:

1'heat A and B separately to 80° C. until complete homogenization,
2—add A to B using a turbomixer at around 75° C.,
3—homogenize,
4—cool to 40° C. and add C using a turbomixer,
5—add D and then, at 25° C., add E then F.

### Example 3

### Demonstration of a Synergy of Action of the Combination of Retinol with Adenosine

The effects of the combination are evaluated with respect to epidermal differentiation and proliferation in the Realskin reconstructed epidermis model.

### Protocol

The Realskin epidermises were incubated in the presence or absence of active agents starting from D 20:
Retinol at 250 μM
Adenosine at 10 μM
250 μM retinol+10 μM adenosine.

The epidermises are analysed histologically and by filaggrin (marker for epidermal differentiation) immunolabelling starting from D 27.

Filaggrin, which is a late differentiation marker, is a product of degradation of profilaggrin, a high MW phosphoprotein contained in the keratohyalin granules. At the moment the keratinocyte transforms into a corneocyte, the keratohyalin granules disaggregate, and the profillagrin undergoes sequential proteolysis which releases oligomers and then monomers of filaggrin.

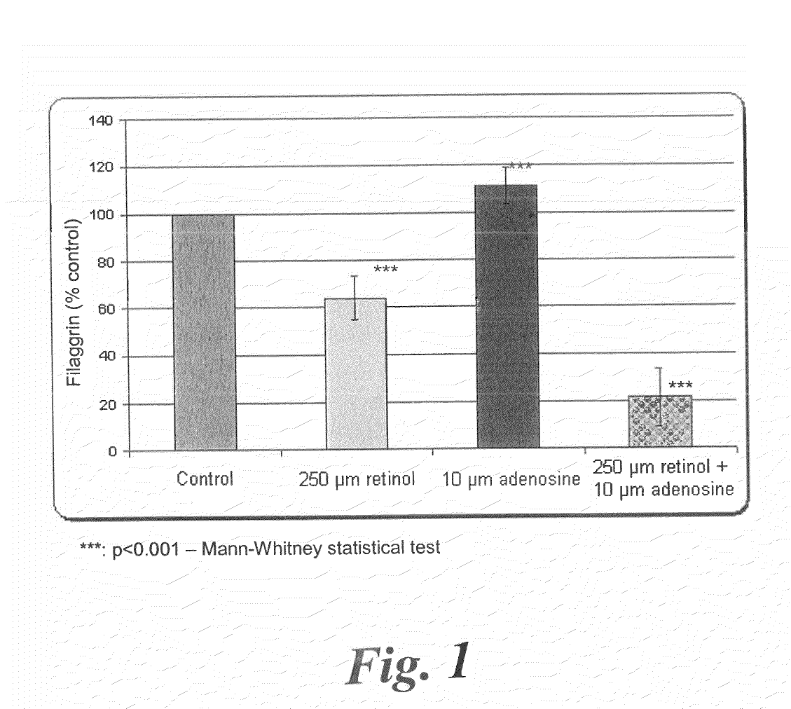

The results arc illustrated in FIG. 1.

They attest to a delay in differentiation on the skins treated with retinol (−36%); increased with adenosine (+11%) and potentiation of the effects of retinol with the combination (−79%).

This test therefore proves that the activity of retinol is reinforced by adenosine, which amounts to evidence of a synergy since the effect observed is significantly greater than the sum of the respective effects of the retinol and of the adenosine considered separately from one another.

Such combinations, and compositions comprising such amounts that act synergistically, are termed synergistic combinations herein. Another way to denote this aspect of the present invention is to term it as synergistic amounts of retinol and adenosine. In this regard, the present invention generally relates to synergistic combinations and amounts of retinoid or retinoid derivative and adenosine-based nonphosphated compound.

### Example 4

### Demonstration of the Effectiveness of a Composition in Accordance with the Invention

This effectiveness was assessed according to two methodologies, namely a cosmeto-clinical method and a characterization by chromasphere.

### 1. Cosmeto-Clinical

The composition in accordance with that of Example 1, but free of EDTA, was tested on 50 women of Caucasian origin between the ages of 45 and 65, having crowsfeet wrinkle/fine lines, wrinkle/fine lines on the forehead, nasal groove, between the eyebrows, ptosis of the lower part of the face and skin tonicity problems.

The composition was applied once a day for 8 weeks.

### Results: Clinical Scoring

The dermatologist evaluated, at T0, T28 and T56 days, using atlases providing information on the state of the wrinkles considered below, at various stages of skin ageing:
forehead wrinkles,
wrinkles under the eye,
nasal groove wrinkles,
ptosis of the lower part of the face, and
crowsfeet wrinkles (inclusion >3)

The results of the change in state of the wrinkles under consideration, as a function of time, are given in Table 1 below.

TABLE 1

| | T28 days/T0 | T56 days/T0 |
|---|---|---|
| Forehead wrinkles | −17% S* | −22% S |
| Wrinkles under the eye | −23% S | −35% S |
| Nasal groove wrinkles | −11% S | −29% S |
| Ptosis of the lower part of the face | −10% S | −18% S |
| Crowsfeet wrinkles | −15% S | −26% S |

*S = significant

These results demonstrate the significant effect of the product on the reduction of all the parameters studied after 28 days of daily use. The effectiveness is further reinforced after 56 days.

### 2. Chromasphere

The chromasphere is a technique for the acquisition of calibrated colour photos using a daylight-type illumination (D65), which is diffuse, homogeneous and reproducible. Combined with a Hitachi HVF22F camera and a colour card, it is the reference tool for measuring colour. It is in particular described in document FR 2 929 344.

Protocol: formulation tested: composition of Example 1, free of EDTA

Methods: Chromasphere (diffuse light):

Parameters: On the chromasphere images (visibility):
Ra profiles (average depth of the profile with Visibility)
Rz profiles (maximum depth of the profile with Visibility)
Histograms: Histos (Area Tᵢ-T₀ with Vistograms)

Experimental scheme: No randomization
T₀: 2 sides nontreated
Tᵢₘₘ: left side nontreated (L)/right side treated (R)
T₂ₘₒₙₜₕₛ: 2 sides treated

Application: Daily for 2 months

Panel: 34 models over the age of 55 (panel average: 66.4±6.3 years old)

Zone: Crowsfoot, inclusion with score >3.3 on the crowsfoot wrinkle atlas

Time: T₀, Tᵢₘₘ, T_{2 months}

As regards the data obtained by virtue of the various analyses, they give account of the APPARENT distribution of the wrinkles (VISIBILITY of the wrinkles). The values are calculated from 2D (.TIFF) colour photos acquired in diffuse light under calibrated and reproducible conditions (conventional chromasphere).

The visibility of the wrinkles was evaluated on the photos acquired on the conventional chromasphere using the “Visibility” analysis software.

The “Visibility” software makes it possible to obtain, after having converted the colour information into levels of grey, the parameters of average (Ra) and maximum (Rz) apparent roughness of 5 vertical parallel bands distributed over the field of the image, from the corner of the eye to the temple. In addition to these values, the means over all the 5 bands are also calculated.

The results are given in Tables 2 and 3 below:

TABLE 2
Immediate effect on the appearance of wrinkles

| | | | | | Time |
|---|---|---|---|---|---|
| VISIBILITY OF WRINKLES | | | T₀ | Tᵢₘₘ | effect |
| Chroma- | Ra | Treated (R) | 4.26 ± 0.99 | 3.79 ± 1.07 | S |
| sphere | Rz | Treated (R) | 30.76 ± 7.69 | 27.38 ± 8.69 | S |

| | | | |
|---|---|---|---|
| Histos | Treated (R) | 625 ± 1070* | S |

*Histograms directly on the (Tᵢₘₘ − T₀), comparison with the reference value 0
Tₒ = before application of the product
Tᵢₘₘ = just after application of the product

On the zone treated, the formulation tested induces a significant immediate effect on the visibility of the wrinkles by chromasphere. This effect is measured on the deepest relief (Rz) and on all the reliefs analysed (Ra). A significant effect is also detected on the difference in the areas in histograms, compared with the reference value “0” (Histos), signifying that the variations in levels of grey around the average plane have decreased between T₀ and Tᵢₘₘ.

TABLE 3
Effect, at 2 months, of application on the appearance of wrinkles

| | | | | | Time |
|---|---|---|---|---|---|
| VISIBILITY OF WRINKLES | | | T₀ | T_{2 mois} | effect |
| Chroma- | Ra | Treated | 4.30 ± 1.00 | 3.95 ± 1.05 | S |
| sphere | | (R and L) | | | |
| | Rz | Treated | 31.33 ± 7.97 | 29.09 ± 8.72 | S |
| | | (R and L) | | | |

| | | | |
|---|---|---|---|
| Histos | Treated | 502 ± 1603 | S |
| | (R and L) | | |

T_{2 months} = after daily application of the product for 2 months

As the long-term effect of the formulation tested, a significant decrease in the visibility of wrinkles is detected on the maximum roughness parameter Rz, the average roughness parameter Ra and the histogram parameter Histos between T₀ and T₂ₘₒₙₜₕₛ.

### Examples 5 and 6

### Influence of the Semicrystalline Polymer with One or More Crystallizable Side Chain(s) on the Antiwrinkle Effectiveness of the Compositions

The following examples of formulations of creams in the form of an O/W emulsion make it possible to compare the influence of the semicrystalline polymer with one or more crystallizable side chain(s) on the antiwrinkle effectiveness of the compositions.

| | | |
|---|---|---|
| | Example 5 | Example 6 |
| Compounds | (Invention) | (Comparative) |

Aqueous phase A:

| | | |
|---|---|---|
| Water | q.s. 100% | q.s. 100% |
| Glycerol | 5% | 5% |
| Adenosine*** | 0.1% | 0.1% |
| Preservatives | 0.65% | 0.65% |
| Ethylenediaminetetraacetic acid, disodium | 0.3% | 0.3% |
| salt | | |
| Sucrose monodipalmitostearate | 1.5% | 1.5% |
| Crosslinked acrylic acid/alkyl acrylate | 0.2% | 0.2% |
| polymer | | |

Fatty phase B1:

| | | |
|---|---|---|
| Fatty substance | 5% | 5% |
| UV screening agents | 15% | 15% |
| Stearic acid | 1.2% | 1.2% |
| Caprylyl glycol | 0.2% | 0.2% |
| Poly(stearyl acrylate)** | 2% | — |

Phase B2:

| | | |
|---|---|---|
| Fragrance | 0.4% | 0.4% |

Phase C1:

| | | |
|---|---|---|
| Cyclohexasiloxane | 3% | 3% |
| Sodium hyaluronate | 0.1% | 0.1% |
| Crosslinked poly(sodium acrylate) | 0.5% | 0.5% |

Phase C2:

| | | |
|---|---|---|
| Cyclohexasiloxane | 2% | 2% |
| Crosslinked polymer of dimethicone and | 3% | 3% |
| dimethicone/vinyl dimethicone | | |

Phase D:

| | | |
|---|---|---|
| Filler | 1% | 1% |

Phase E:

| | | |
|---|---|---|
| Retinol* | 0.07% | 0.07% |

*Retinol 10 SU sold by BASF (expressed as active material)
**Intelimer ® IPA 13-1 from the company Air Product and Chemicals
***Adenosine sold by Pharma Waldhof GmbH

### Procedure:

1—heat A and B1 separately to 80° C. until complete homogenization,
2—add A to B1+B2 using a turbomixer at around 75° C.,
3—homogenize, add C1 at 40° C. using a turbomixer,
4—then C2 at 30° C.,
5—add D and then, at 25° C., add E with nitrogen inerting.

### Evaluation

The creams obtained are evaluated on a panel of 6 women, with regard to the immediate cosmetic effects observed.

On the first day, an amount of 0.30 ml of the composition of Example 5 according to the invention is applied to a single half of the face of each of the six women, and in particular in the zone located below the eye.

A photograph is taken just after the application.

The following day, an amount of 0.30 ml of the composition of comparative Example 6 is applied to the same single half of the face than the one on the first day for each of the six women.

A photograph is taken just after the application.

The application of the compositions to the halves of faces is randomized; in other Words, for the first person the selected half of face is the right half of face, for the second person the selected half of face is the left half of face, etc.

The photographs taken respectively after the application of the composition of Example 5 and of the composition of Example 6 on the different halves of faces are compared.

### Results

From the results of the study, a reduction in the wrinkles under the eye is observed after application of the cream of Example 5 according to the invention. These wrinkles appear to be much less pronounced. Such a reduction in wrinkles under the eye is not observed after application of the cream of Example 6, which is not part of the invention.

Thus, the presence of the semicrystalline polymer with one or more crystallizable side chain(s) makes it possible to significantly reinforce the antiwrinkle effectiveness of the compositions according to the invention, relative to a composition devoid of said polymer.

The above written description of the invention provides a manner and process of making and using it such that any person skilled in this art is enabled to make and use the same, this enablement being provided in particular for the subject matter of the appended claims, which make up a part of the original description.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. The term “mentioned” notes exemplary embodiments, and is not limiting to certain species. As used herein the words “a” and “an” and the like carry the meaning of “one or more.”

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention; and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

The invention compositions and combinations are preferably used by human subjects desirous of the benefits noted herein, subjects “in need of” these benefits. Such subjects are typically suffering from one or more of the conditions, symptoms, etc. addressed by the present invention, such as by self diagnosis or cosmetician or medical diagnosis, or are at recognized and appreciated risk of developing such conditions, etc. and who intentionally use the invention methods, compositions and combinations to treat, address, combat, prevent, etc. the effects of such conditions, etc. The application also clearly describes and supports the simple application of the invention composition and combination on the skin and its integuments regardless of any purpose or intent.

1. A composition, comprising, in a physiologically acceptable medium, a retinoid or retinoid derivative, an adenosine-based nonphosphated compound, and a semicrystalline polymer with one or more crystallizable side chain(s).

2. The composition according to claim 1, wherein said retinoid or retinoid derivative is chosen from retinol, retinal, retinoic. acid, and an ester of retinol with a C₂-C₂₀ acid.

3. The composition according to claim 1, comprising 0.005% to 5% by weight of said retinoid or retinoid derivative, relative to the total weight of the composition.

4. The composition according to claim 1, wherein the adenosine-based nonphosphated compound is adenosine.

5. The composition according to claim 1, comprising 0.0001% to 5% by weight of adenosine-based nonphosphated compound relative to the total weight of the composition.

6. The composition according to claim 1, wherein the semicrystalline polymer with one or more crystallizable side chain(s) is chosen from homopolymers resulting from the polymerization of at least one monomer with a crystallizable side chain chosen from saturated C₁₀ to C₃₀ alkyl (meth)acrylates represented by the formula: 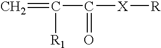
in which R₁ is H or CH₃, R represents a C₁₀ to C₃₀ alkyl group, and X represents O.

7. The composition according to claim 1, wherein the semicrystalline polymer with one or more crystallizable side chain(s) is chosen from homopolymers resulting from the polymerization of a monomer with a crystallizable chain chosen from C₁₄-C₂₄ alkyl acrylates and C₁₄-C₂₄ alkyl methacrylates.

8. The composition according to claim 1, wherein the semicrystalline polymer with one or more crystallizable side chain(s) is chosen from a homopolymer of stearyl acrylate and a homopolymer of behenyl acrylate.

9. The composition according to claim 1, comprising at least 0.6% by weight of semicrystalline polymer with one or more crystallizable side chain(s) relative to the total weight of the composition.

10. The composition according to claim 1, comprising retinol and/or retinyl palmitate, an adenosine-based nonphosphated compound, and a C₁₀-C₃₀ alkyl (meth)acrylate homopolymer.

11. The composition according to claim 1, further comprising at least one additional polymer which is not a semicrystalline polymer with one or more crystallizable side chain(s) and is chosen from acrylic polymers, hydrophobically modified polysaccharides and fatty acid esters of polyols.

12. The composition according to claim 1, further comprising at least one liquid fatty phase.

13. The composition according to claim 1, further comprising at least one supplementary cosmetic active agent chosen from antiwrinkle active agents other than adenosine, retinol and derivatives thereof, UV screening agents, desquamating agents, antioxidants, moisturizers, active agents for stimulating the synthesis of dermal and/or epidermal macromolecules, dermodecontracting agents, and mixtures thereof.

14. The composition according to claim 1, comprising a synergistic combination of said retinoid or retinoid derivative and said adenosine-based nonphosphated compound.

15. The composition according to claim 1, comprising a synergistic combination of retinol and adenosine.

16. A method, comprising applying the composition of claim 1 to human skin.

17. A method for preventing and/or treating the signs of skin ageing and/or sagging skin, comprising applying the composition of claim 1 to human skin in need thereof.

18. A method for reducing and/or smoothing out skin imperfections and/or for obtaining skin which is smoother, more homogeneous, firmer, more tonic and more elastic, comprising applying the composition of claim 1 to human skin in need thereof.

19. A method for preventing and/or treating the cutaneous signs of ageing, comprising applying the composition of claim 1 to human skin in need thereof.
